SEQUENCE LISTING (69 sequences)

SEQ ID NO: 1 - protein, 624 aa, artificial sequence
  Recombinant immunotoxin, antibody CDR, linker or toxin
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 2 - protein, 625 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 3 - protein, 632 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHRRRKRSLDTSAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 4 - protein, 633 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGTRHRQPRGWEQLAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 5 - protein, 631 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGNRVRRSVGAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 6 - protein, 634 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSRRKRAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 7 - protein, 213 aa, artificial sequence
  Recombinant immunotoxin, antibody CDR, linker or toxin
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 610 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSAETVQYYNSYSDASIASCAFVDSGKDKIDKTKLVTYTSRLAASPAYQKVVGVGLKTAAGSIVPYVRLDMDNTGKGIHFNATKLSDSSAKLAAVLKTTVSMTEAQRTQLYMEYIKGIENRSAQFIWDWWRTGKAPA

SEQ ID NO: 9 - protein, 605 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSAPIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 10 - protein, 608 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 11 - protein, 615 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHRRRKRSLDTSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 12 - protein, 616 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGTRHRQPRGWEQLADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 13 - protein, 587 aa, artificial sequence
  Recombinant immunotoxin, antibody CDR, linker or toxin
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 14 - protein, 213 aa, artificial sequence; identical to SEQ ID NO: 7
  Recombinant immunotoxin, antibody CDR, linker or toxin

SEQ ID NO: 15 - protein, 594 aa, artificial sequence
  Recombinant immunotoxin, antibody CDR, linker or toxin
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHRRRKRSLDTSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 16 - protein, 213 aa, artificial sequence; identical to SEQ ID NO: 7
  Recombinant immunotoxin, antibody CDR, linker or toxin

SEQ ID NO: 17 - protein, 583 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 18 - protein, 218 aa, artificial sequence
  Recombinant immunotoxin, antibody CDR, linker or toxin
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 590 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHRRRKRSLDTSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 20 - protein, 218 aa, artificial sequence; identical to SEQ ID NO: 18
  Recombinant immunotoxin, antibody CDR, linker or toxin

SEQ ID NO: 21 - protein, 591 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGTRHRQPRGWEQLADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 22 - protein, 218 aa, artificial sequence; identical to SEQ ID NO: 18
  Recombinant immunotoxin, antibody CDR, linker or toxin

SEQ ID NO: 23 - protein, 356 aa, artificial sequence
  Recombinant immunotoxin, antibody CDR, linker or toxin
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 24 - protein, 446 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 356 aa, artificial sequence; identical to SEQ ID NO: 23
  Recombinant immunotoxin, antibody CDR, linker or toxin

SEQ ID NO: 26 - protein, 583 aa, artificial sequence; identical to SEQ ID NO: 17
  Recombinant immunotoxin, antibody CDR, linker or toxin

SEQ ID NO: 27 - protein, 157 aa, artificial sequence
  Recombinant immunotoxin, antibody CDR, linker or toxin
APSPLDARATWTCINQQLNPKTNKWEDKRLLYNQAKAESNSHHAPLSDGKTGSSYPHWFTNGYDGNGKLIKGRTPIKFGKADCDRPPKHSQNGMGKDDHYLLEFPTFPDGHDYKFDSKKPKEDPGPARVIYTYPNKVFCGIVAHQRGNQGDLRLCSH

SEQ ID NO: 28 - protein, 608 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSADTVICEPHQDTETGKIKKFKVDVGVAEDQAKKAGLITGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 29 - protein, 608 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSADTVICEPRQDTETGKIKKFKVDVGVAEDQAKKAGLITGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 30 - protein, 608 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSADTVICEPHQDTETGKIKKFTVDVGVAEDQAKKAGLTTGKSGDPHRYMNGDNINFGIHNCDKEGAILWEYPIYWVGKKAEWKKDEKTDRQPGGPTPLRVVYANNNGNIVYCGVMTHAVVKSNNQGEKFFLKCT

SEQ ID NO: 31 - protein, 607 aa, artificial sequence
  Recombinant immunotoxin, antibody CDR, linker or toxin
EVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDIHGYLNWYQQKPDGTVKLLIYYTSILHSGVPSRFSGSGSGTDYSLTISNLEQEDFATYFCQQGNTLPWTFGGGTKLEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSRRKRASKESRAKKFQRQHMDSDSSPSSSSTYCNQMMRRRSMTQGDCKPVNTFVHEPLVDVQNVCFQEKVTCKDGQGNCYKSNSAMHITDCRLTADSDYPNCAYRTSPKERHIIVACEGSPYVPVHFDASV

SEQ ID NO: 32 - protein, 625 aa, artificial sequence; identical to SEQ ID NO: 2
  Recombinant immunotoxin, antibody CDR, linker or toxin

SEQ ID NO: 33 - protein, 472 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 472 aa, artificial sequence; identical to SEQ ID NO: 33
  Recombinant immunotoxin, antibody CDR, linker or toxin

SEQ ID NO: 35 - protein, 606 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSRRKRASKESRAKKFQRQHMDSDSSPSSSSTYCNQMMRRRSMTQGDCKPVNTFVHEPLVDVQNVCFQEKVTCKDGQGNCYKSNSAMHITDCRLTADSDYPNCAYRTSPKERHIIVACEGSPYVPVHFDASV

SEQ ID NO: 36 - protein, 598 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSGGGGSGGGGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKASTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSQDNSRYTHFLTQHYDAKPQGRDDRYCESIMRRRGLTSPCKDINTFIHGNKRSIKAICENKNGNPHRENLRISKSSFQVTTCKLHRRSPWPPCQYRATAGFRNVVVACENGLPVHLDQSIFRRP

SEQ ID NO: 37 - protein, 625 aa, artificial sequence; identical to SEQ ID NO: 2
  Recombinant immunotoxin, antibody CDR, linker or toxin

SEQ ID NO: 38 - protein, 633 aa, artificial sequence; identical to SEQ ID NO: 4
  Recombinant immunotoxin, antibody CDR, linker or toxin

SEQ ID NO: 39 - protein, 608 aa, artificial sequence; identical to SEQ ID NO: 10
  Recombinant immunotoxin, antibody CDR, linker or toxin

SEQ ID NO: 40 - protein, 600 aa, artificial sequence
  Recombinant immunotoxin, antibody CDR, linker or toxin
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSAVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 41 - protein, 218 aa, artificial sequence; identical to SEQ ID NO: 18
  Recombinant immunotoxin, antibody CDR, linker or toxin

SEQ ID NO: 42 - protein, 583 aa, artificial sequence; identical to SEQ ID NO: 17
  Recombinant immunotoxin, antibody CDR, linker or toxin

SEQ ID NO: 43 - protein, 218 aa, artificial sequence; identical to SEQ ID NO: 18
  Recombinant immunotoxin, antibody CDR, linker or toxin

SEQ ID NO: 44 - protein, 12 aa, artificial sequence
  Recombinant immunotoxin, antibody CDR, linker or toxin
TRHRQPRGWEQL

SEQ ID NO: 45 - protein, 10 aa, artificial sequence
  Recombinant immunotoxin, antibody CDR, linker or toxin
AGNRVRRSVG

SEQ ID NO: 46 - protein, 11 aa, artificial sequence
  Recombinant immunotoxin, antibody CDR, linker or toxin
HRRRKRSLDTS

SEQ ID NO: 47 - protein, 130 aa, artificial sequence
  Recombinant immunotoxin, antibody CDR, linker or toxin
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 48 - protein, 133 aa, artificial sequence
  Recombinant immunotoxin, antibody CDR, linker or toxin
ADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 49 - protein, 133 aa, artificial sequence
  Recombinant immunotoxin, antibody CDR, linker or toxin
ADTVICEPHQDTETGKIKKFKVDVGVAEDQAKKAGLTTGKSGYPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 50 - protein, 123 aa, artificial sequence
  Recombinant immunotoxin, antibody CDR, linker or toxin
QDNSRYTHFLTQHYDAKPQGRDDRYCESIMRRRGLTSPCKDINTFIHGNKRSIKAICENKNGNPHRENLRISKSSFQVTTCKLHRRSPWPPCQYRATAGFRNVVVACENGLPVHLDQSIFRRP

SEQ ID NO: 51 - protein, 133 aa, artificial sequence
  Recombinant immunotoxin, antibody CDR, linker or toxin
ADTVICEPHQDTETGKIKKFKVDVGVAEDQAKKAGLITGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 52 - protein, 133 aa, artificial sequence
  Recombinant immunotoxin, antibody CDR, linker or toxin
ADTVICEPRQDTETGKIKKFKVDVGVAEDQAKKAGLITGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 53 - protein, 133 aa, artificial sequence
  Recombinant immunotoxin, antibody CDR, linker or toxin
ADTVICEPHQDTETGKIKKFTVDVGVAEDQAKKAGLTTGKSGDPHRYMNGDNINFGIHNCDKEGAILWEYPIYWVGKKAEWKKDEKTDRQPGGPTPLRVVYANNNGNIVYCGVMTHAVVKSNNQGEKFFLKCT

SEQ ID NO: 54 - protein, 135 aa, artificial sequence
  Recombinant immunotoxin, antibody CDR, linker or toxin
AETVQYYNSYSDASIASCAFVDSGKDKIDKTKLVTYTSRLAASPAYQKVVGVGLKTAAGSIVPYVRLDMDNTGKGIHFNATKLSDSSAKLAAVLKTTVSMTEAQRTQLYMEYIKGIENRSAQFIWDWWRTGKAPA

SEQ ID NO: 55 - protein, 150 aa, artificial sequence
  Recombinant immunotoxin, antibody CDR, linker or toxin
AVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 56 - protein, 150 aa, artificial sequence
  Recombinant immunotoxin, antibody CDR, linker or toxin
AVTWTCLNTQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENTGELKLCSH

SEQ ID NO: 57 - protein, 128 aa, artificial sequence
  Recombinant immunotoxin, antibody CDR, linker or toxin
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 58 - protein, 10 aa, artificial sequence
  Recombinant immunotoxin, antibody CDR, linker or toxin
GYTFSSYWIE

SEQ ID NO: 59 - protein, 18 aa, artificial sequence
  Recombinant immunotoxin, antibody CDR, linker or toxin
GEILPGGGDTNYNEIFKG

SEQ ID NO: 60 - protein, 10 aa, artificial sequence
  Recombinant immunotoxin, antibody CDR, linker or toxin
TRRVPIRLDY

SEQ ID NO: 61 - protein, 15 aa, artificial sequence
  Recombinant immunotoxin, antibody CDR, linker or toxin
KASQSVDYEGDSFLN

SEQ ID NO: 62 - protein, 7 aa, artificial sequence
  Recombinant immunotoxin, antibody CDR, linker or toxin
AASNLES

SEQ ID NO: 63 - protein, 10 aa, artificial sequence
  Recombinant immunotoxin, antibody CDR, linker or toxin
CQQSNEDPLT

SEQ ID NO: 64 - protein, 9 aa, artificial sequence
  Recombinant immunotoxin, antibody CDR, linker or toxin
YTFTSYNMH

SEQ ID NO: 65 - protein, 14 aa, artificial sequence
  Recombinant immunotoxin, antibody CDR, linker or toxin
WIGAIYPGNGDTSY

SEQ ID NO: 66 - protein, 13 aa, artificial sequence
  Recombinant immunotoxin, antibody CDR, linker or toxin
RSTYYGGDWYFNV

SEQ ID NO: 67 - protein, 7 aa, artificial sequence
  Recombinant immunotoxin, antibody CDR, linker or toxin
SSVSYIH

SEQ ID NO: 68 - protein, 11 aa, artificial sequence
  Recombinant immunotoxin, antibody CDR, linker or toxin
PWIYATSNLAS

SEQ ID NO: 69 - protein, 8 aa, artificial sequence
  Recombinant immunotoxin, antibody CDR, linker or toxin
QQWTSNPP

(19) United States
(12) Patent Application Publication
(10) Pub. No.: US 2023/0310557 A1
(43) Pub. Date: Oct. 5, 2023

(54) RECOMBINANT IMMUNOTOXIN COMPRISING A RIBOTOXIN OR RNASE

(71) Applicant: ATB Therapeutics, Aye (BE)

(72) Inventors: Sébastien MERCX, Aye (BE); Max HOURY, Aye (BE); Bertrand MAGY, Aye (BE)

(21) Appl. No.: 18/041,770

(22) PCT Filed: Aug. 17, 2021
(86) PCT No.: PCT/EP2021/072859

§ 371 Date: Feb. 15, 2023

(30) Foreign Application Priority Data
Aug. 17, 2020 (EP) 20191417.3
Aug. 17, 2020 (EP) 20191419.9
Feb. 10, 2021 (EP) 21156276.4
Feb. 12, 2021 (EP) 21156837.3
Jul. 19, 2021 (EP) 21186435.0

(51) Int. Cl. A61K 38/46; C12N 9/22; C07K 16/28

(52) U.S. Cl. CPC A61K 38/465; C12N 9/22; C07K 16/2887; C07K 16/2803; C07K 2319/00

(57) ABSTRACT

The present invention relates to a binder-toxin fusion protein comprising at least one protein binder selected from the group consisting of
an antibody
an antibody fragment or derivative retaining target binding capacity, or
an antibody mimetic,
   a ribotoxin or -protoxin, and optionally, a peptide linker connecting a) and b) and/or a cleavable domain comprised in the protoxin (FIG. 1)

### FIELD OF THE INVENTION

The present application relates to the field of a binder-toxin fusion proteins

### BACKGROUND

Conjugates combining a target binder and a toxin have been developed forty years ago and now represent a major hope to fight cancer. These conjugates are mainly represented by the class of Antibody-Drug-Conjugates (ADC), consisting of a monoclonal antibody chemically conjugated to a chemical cytotoxic agent via a linker. These drugs combine the specificity of monoclonal antibodies to target cancer cells with the high toxic potency of the payload, to kill targeted cells, while sparing healthy tissues.

There is still a need for new such entities to provide better treatment options for different tumor types. It is hence one object of the present invention to provide such new entities.

It is one further object of the present invention to provide alternative or even better treatment options for cancer patients These and further objects are met with methods and means according to the independent claims of the present invention. The dependent claims are related to specific embodiments.

### SUMMARY OF THE INVENTION

The methodologies used in the conception and reduction to practice of this invention are disclosed in PCT application PCT/EP2020/054263, the content of which is incorporated herein by reference in its entirety. The definitions and embodiments disclosed therein form part of the present disclosure. For clarity, the text of PCT application PCT/EP2020/054263 is appended to this application and forms part of its disclosure.

### EMBODIMENTS OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

The embodiments of the present invention are shown in the claims.

According to one embodiment, a binder-toxin fusion protein comprising anisoplin or an active fragment thereof is provided. Preferably, the binder-toxin fusion protein comprises a toxin sequence according to SEQ ID NO 48 or 49, or a homologue thereof having at least 66% sequence identity with SEQ ID NO 48 or 49.

According to one embodiment, a binder-toxin fusion protein comprising an anisoplin homologue or an active fragment thereof is provided. Preferably, the binder-toxin fusion protein comprises a toxin sequence according to SEQ ID NO 51, 52 or 53, or a homologue thereof having at least 66% sequence identity with SEQ ID NO 51, 52 or 53.

Anisoplin is a fungal ribotoxin produced, in nature by the entomopathogenic fungus Metarhizium anisopliae. M. anisopliae was first employed in the late 1800s for biological control of wheat-grain beetles. Since then, biopesticides based on this fungus have greatly evolved. Recently, M. anisopliae has also become an interesting and promising alternative for the control of adult malaria vectors like the Anopheles gambiae mosquito as the appearance of resistance to insecticides hampers the efforts to control the disease.

Anioplin has so far not been described in the context of antitumor treatments, nor as a toxin component in a binder-toxin fusion protein. The inventors have for the first explored the potential of anisoplin in these contexts and have surprisingly found that the toxin has excellent characteristics rendering it suitable for these applications.

In several embodiments, the toxin sequence has a sequence identity of ≥67%; ≥68%; ≥69%; ≥70%; ≥71%; ≥72%; ≥73%; ≥74%; ≥75%; ≥76%; ≥77%; ≥78%; ≥79%; ≥80%; ≥81%; ≥82%; ≥83%; ≥84%; ≥85%; ≥86%; ≥87%; ≥88%; ≥89%; ≥90%; ≥91%; ≥92%; ≥93%; ≥94%; ≥95%; ≥96%; ≥97%; ≥98%; ≥99%, and most preferably 100% with SEQ ID NO 48 or 49.

According to one embodiment, the protein binder is selected from the group consisting of
an antibody
an antibody fragment or derivative retaining target binding capacity, or
an antibody mimetic.

According to one embodiment, the binder-toxin fusion protein comprises a peptide linker connecting the binder, or a domain thereof, with the toxin, or with a cleavable domain comprised in the toxin.

According to several embodiments of the binder-toxin fusion protein
the peptide linker or the cleavable domain is specifically or non-specifically cleavable by an enzyme expressed by a mammalian cell, or an enzyme that is produced by a mammalian host, and/or
the peptide linker or the cleavable domain is not cleavable by an enzyme expressed by a plant cell, or an enzyme that is produced by a plant host, and/or
the binder-toxin fusion protein is expressed in a transfected plant cell or transfected whole plant.

The skilled person has a bunch of routine methods at hand to check whether the condition that peptide linker or the cleavable domain in the protoxin is not cleavable by an enzyme expressed by a plant cell, or an enzyme that is produced by a plant host, is met. See e.g., Wilbers et al (2016). Also, the skilled person can check with routine methods whether the peptide linker or the cleavable domain is specifically or non-specifically cleavable by an enzyme expressed by a mammalian cell, or an enzyme that is produced by a mammalian host,

According to one embodiment, the protein binder binds to human CD20 or human CD79B.

According to further aspects of the invention, a binder-toxin fusion protein comprising at least:
a) one protein binder selected from the group consisting of
   an antibody
   an antibody fragment or derivative retaining target binding capacity, or
   an antibody mimetic,
b) a RNAse, a ribotoxin or a respective protoxin, and
c) optionally, a peptide linker the binder, or a domain thereof, with the toxin, or a cleavable domain comprised in the protoxin.

According to one aspect, such binder-toxin fusion protein is one of the formats selected from the group consisting of
(scFv-FC)-(linker)-toxin (dimer)
tetramer of two HC and two LC-(linker)-toxin
tetramer of two LC and two HC-(linker)-toxin, or
tetramer of two LC-(linker)-toxin and two HC-(linker)-toxin

wherein the linker is optional.

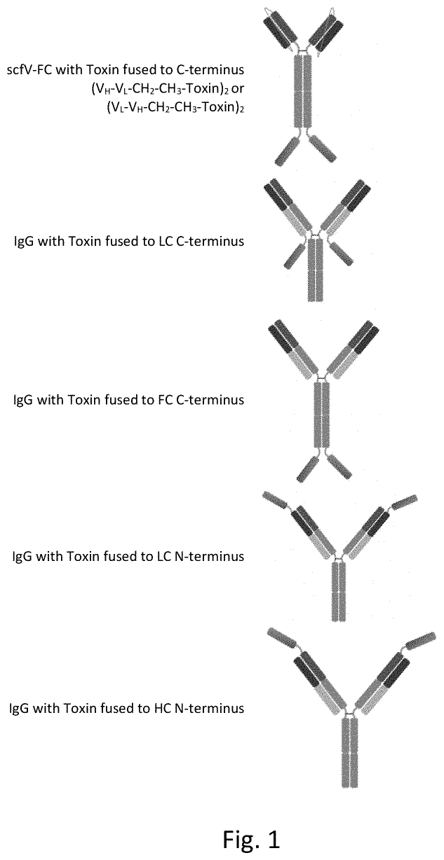

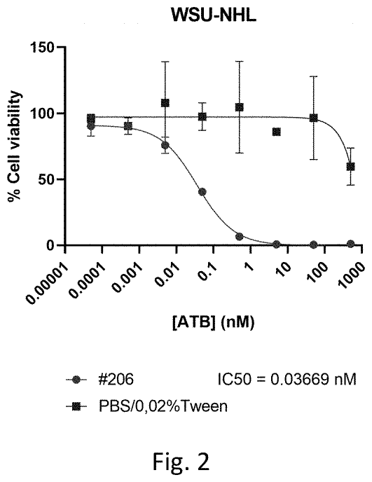

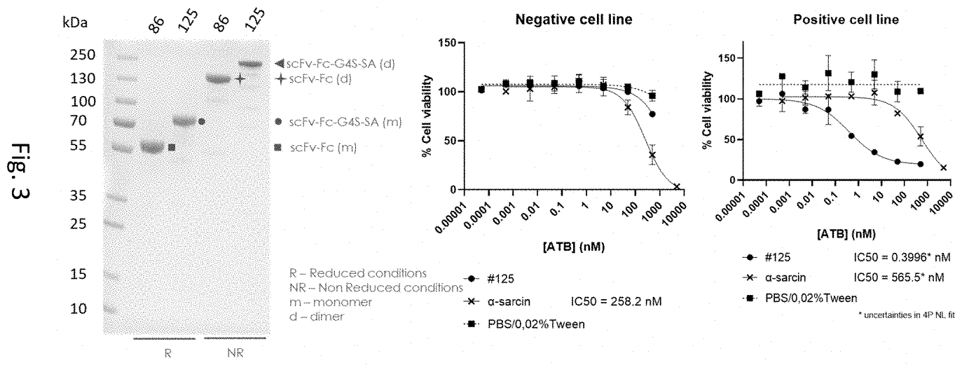

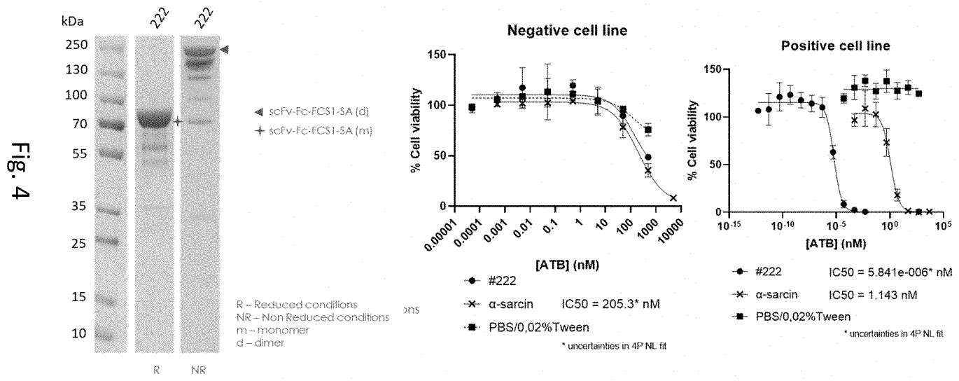

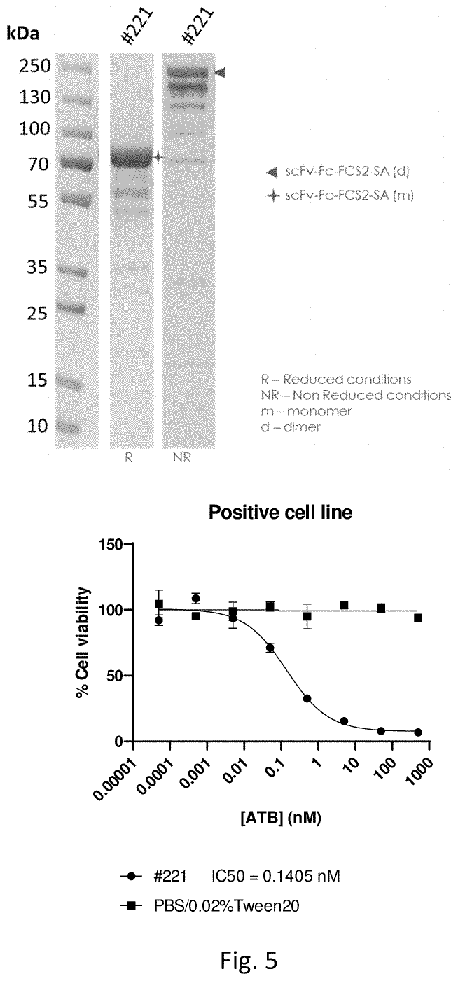

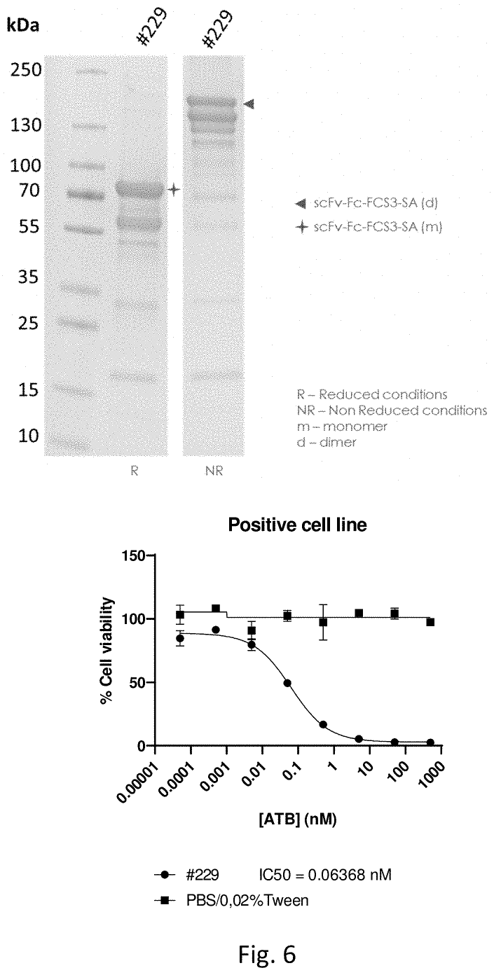

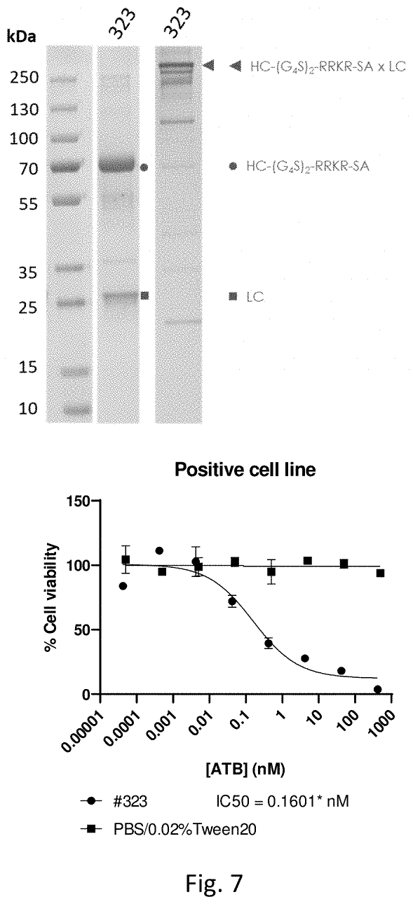

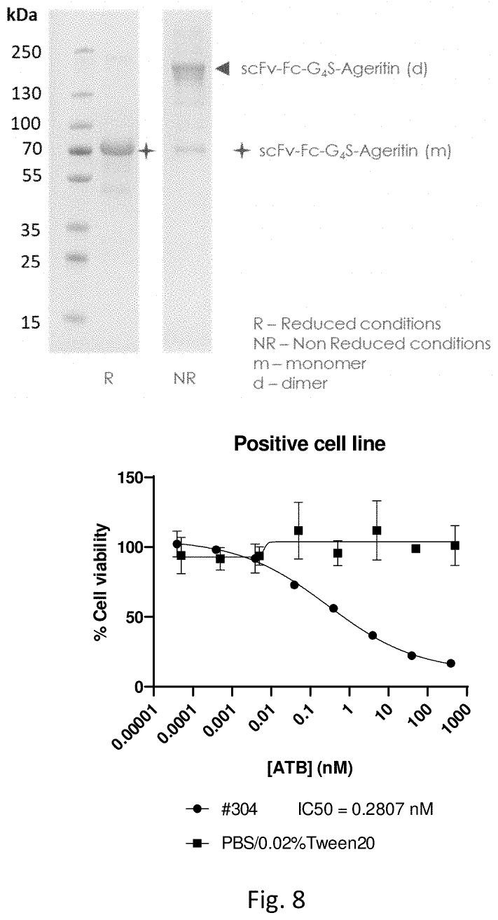

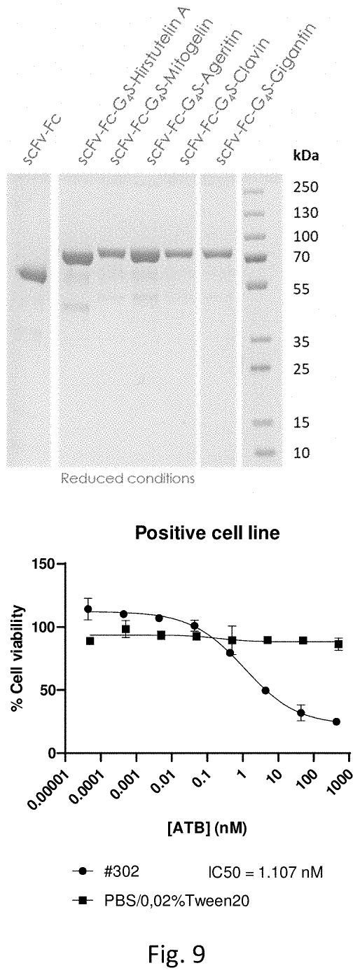

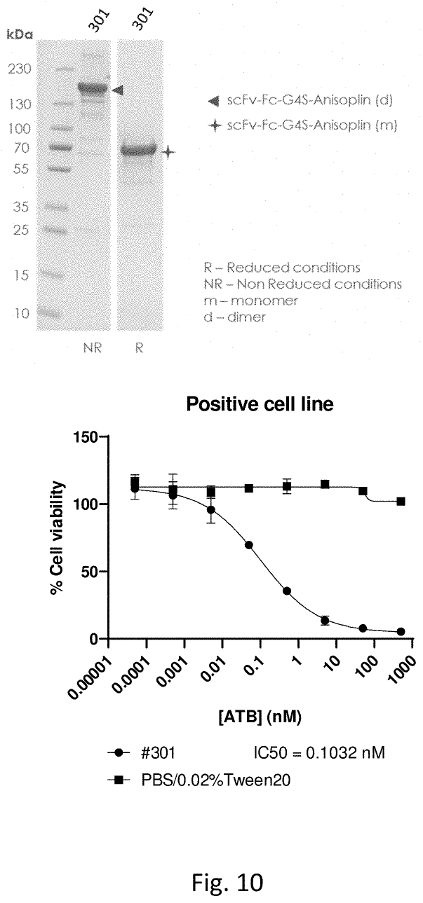

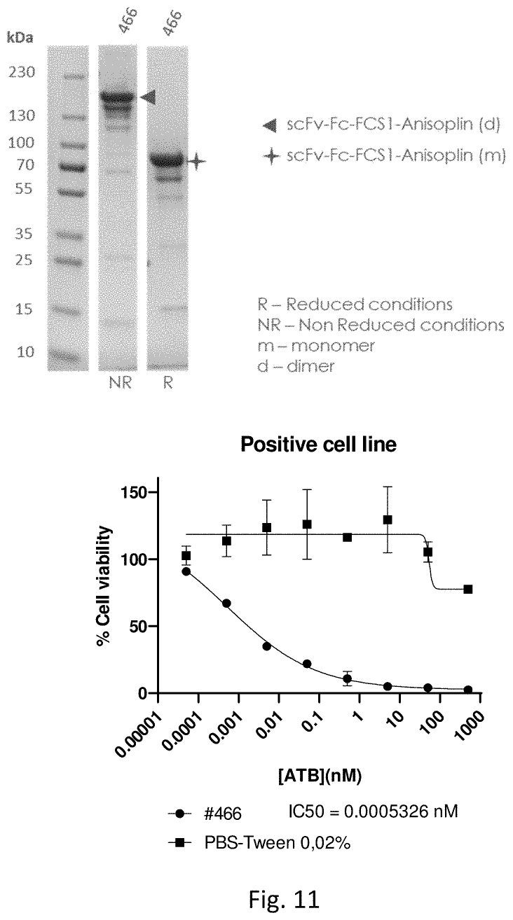

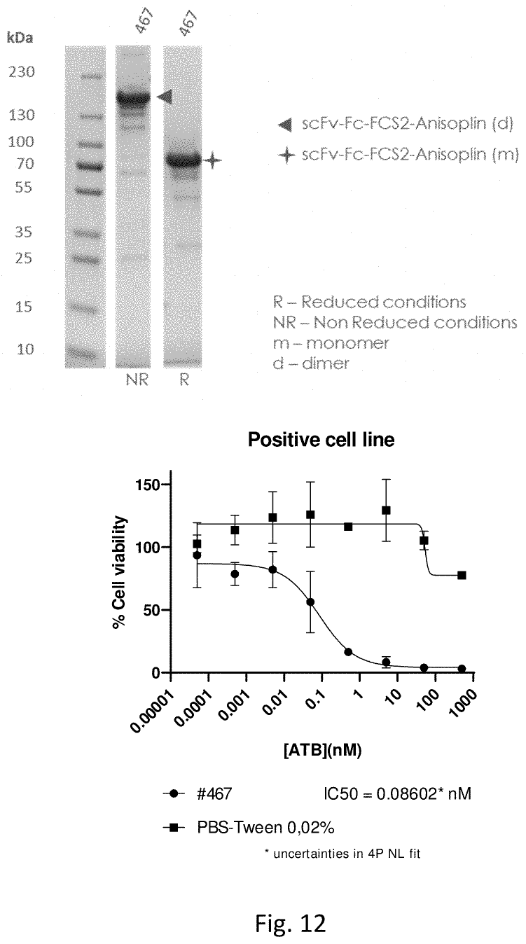

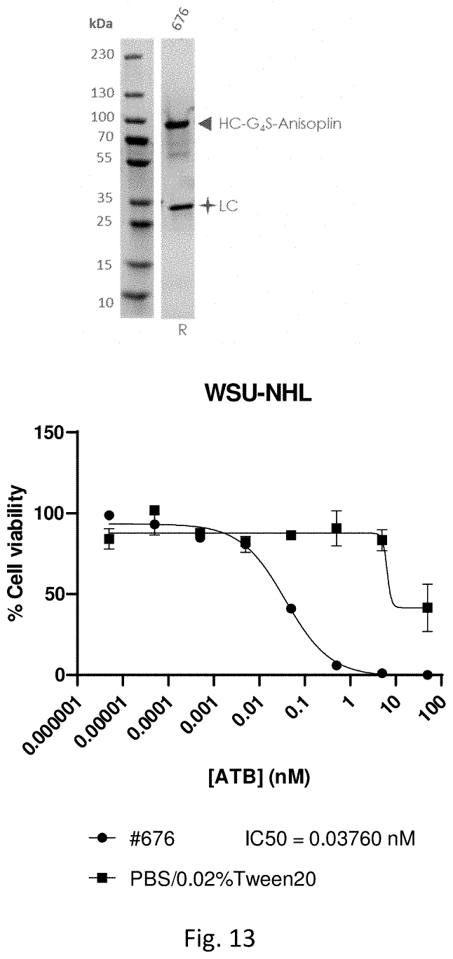

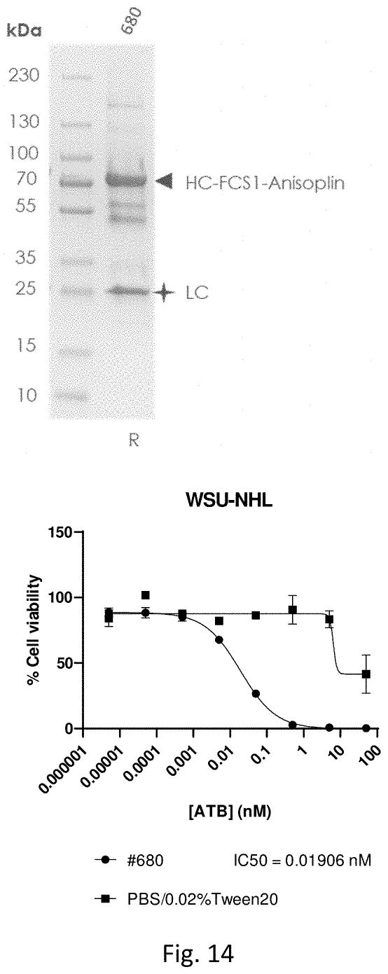

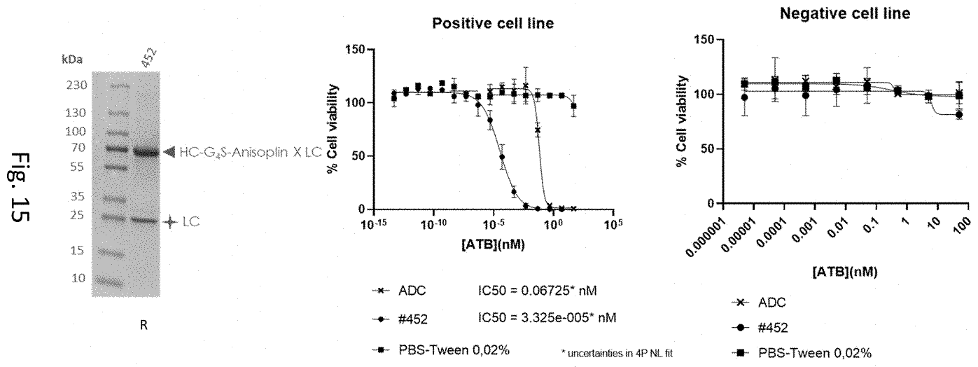

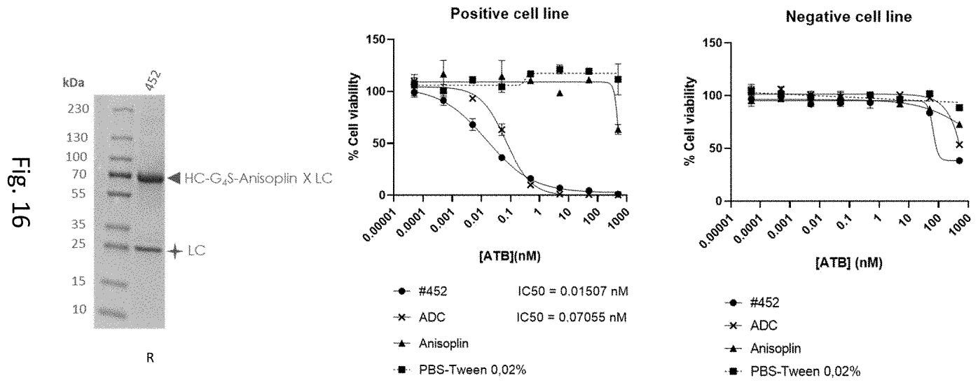

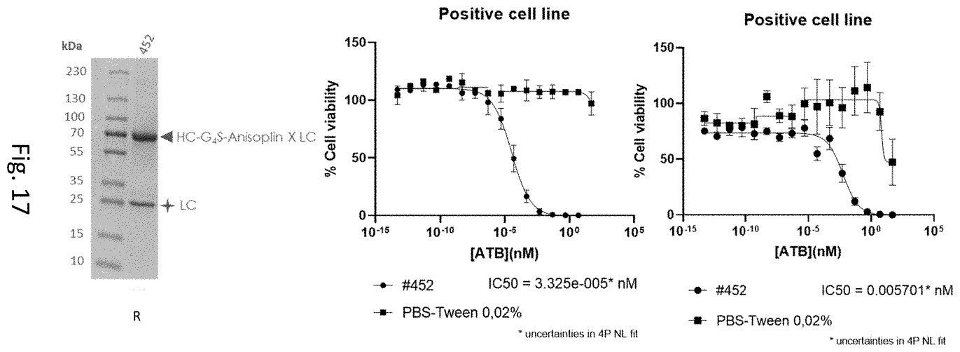

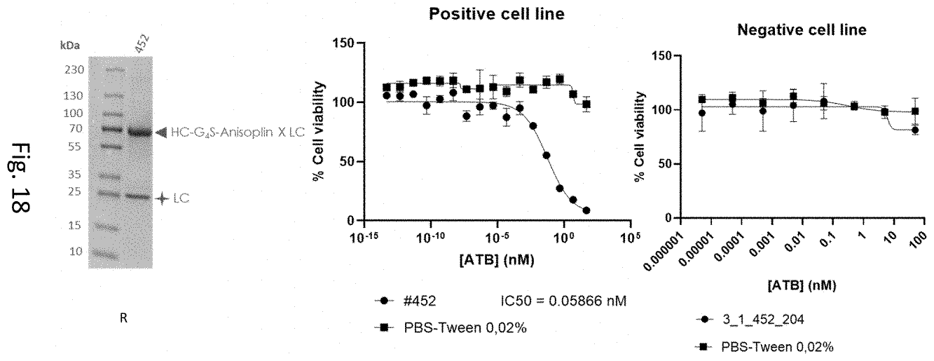

FIG. 1 shows a selection of possible binder-toxin fusion protein formats.
CH₃=heavy chain constant domain 3
CH₂=heavy chain constant domain 2
VL=light chain variable domain
VH=heavy chain variable domain
FC=antibody FC domain
LC=light chain
HC=heavy chain

According to further aspects,
the peptide linker or the cleavable domain in the protoxin is specifically or non-specifically cleavable by an enzyme expressed by a mammalian cell, or an enzyme that is produced by a mammalian host, and/or
the peptide linker or the cleavable domain in the protoxin is not cleavable by an enzyme expressed by a plant cell, or an enzyme that is produced by a plant host.

According to one aspect, such binder-toxin fusion protein is expressed in a transfected plant cell or transfected whole plant.

According to one aspect, the protein binder in such binder-toxin fusion protein binds to human CD20 or human CD79B.

CD79b (B-cell antigen receptor complex-associated protein 0-chain) is a surface protein and involved in the humoral immune response. CD79b is produced by B cells. It binds to CD79a and is linked to it by disulfide bridges Two of these heterodimers bind to membrane-bound antibodies of subtypes mIgM or mIgD to form the B cell receptor (BCR) to which antigens bind. CD79b enhances the phosphorylation of CD79a. Following antigen binding, the antigen-antibody BCR is endocytosed. CD79b is glycosylated. It has an ITAM motif intracellularly that binds and is phosphorylated by the protein kinases Syk or Lyn following activation of the BCR

The full sequence of CD79b has for the first time been disclosed by Hashimoto et al. Immunogenetics. 1994; 40(2):145-149. Protein binders to CD79B have been described in the art. The first antibody (murine) against CD79b is called SN8, and has been published by Okazaki et al., Blood, 81:84-94 (1993)). Polson et al., Blood. 2007; 110(2):616-623 have discussed the possibility to make Antibody drug conjugate (ADCs) or recombinant immunotoxins against CD79b. The first humanized anti CD79b antibody (Polatuzumab) is disclosed in U.S. Pat. No. 8,545,850. In this patent, an ADC consisting of MMAE linked to Polatuzumab is also disclosed.

B-lymphocyte antigen CD20 or is expressed on the surface of all B-cells beginning at the pro-B phase. In humans CD20 is encoded by the MS4A1 gene. The protein has no known natural ligand and its function is to enable optimal B-cell immune response, specifically against T-independent antigens. It is suspected that it acts as a calcium channel in the cell membrane. CD20 is induced in the context of microenvironmental interactions by CXCR4/SDF1 (CXCL12) chemokine signaling and the molecular function of CD20 has been linked to the signaling propensity of B-cell receptor (BCR) in this context.

CD20 is the target of the monoclonal antibodies rituximab, ocrelizumab, obinutuzumab, ofatumumab, ibritumomab tiuxetan, tositumomab, and ublituximab, which are all active agents in the treatment of all B cell lymphomas, leukemias, and B cell-mediated autoimmune diseases. All these antibodies are well described in the prior literature, including their sequences, and shall be deemed to be disclosed in the context of the present invention.

The term “ribotoxin”, as used herein, relates to a group of extracellular ribonucleases (RNases) secreted by fungi. Their most notable characteristic is their extraordinary specificity. They inactivate ribosomes by cutting a single phosphodiester bond of the rRNA that is found in a universally conserved sequence. This cleavage leads to cell death by apoptosis. However, since they are extracellular proteins, they must first enter the cells that constitute their target to exert their cytotoxic action. This entry constitutes the rate-determining step of their action.

All known ribotoxins are proteins of between 130 and 150 amino acids that share at least two different elements of ordered secondary structure: a β-sheet, where the active center is located, and a short α-helix. The structural arrangement is very similar to that of other extracellular fungal RNases, which are not toxic, and constitute a family whose best known representative is the RNase T1 of Aspergillus oryzae. This explains why ribotoxins are considered the toxic representatives of the group. The observation of their three-dimensional structures reveals their functional differences in terms of toxicity, since ribotoxins present unordered, positively charged long loops, which are much shorter, and negatively charged, in their non-toxic “relatives”. These ribotoxin bonds are responsible for recognition of both the negatively charged acid phospholipids that facilitate their entry into cells, and the ribosome-specific features that allow them to cause inactivation.

Ribotoxins cleave RNA following a general acid-base mechanism shared by all the extracellular fungal RNases so far characterized, regardless of their toxicity. Using dinucleosides, such as GpA, it has been demonstrated that the breakage of the phosphodiester bond 3′-5′ of the substrate takes place through the formation of a cyclic intermediate that becomes the corresponding derivative 3′-monophosphate, the final product of the reaction. It is a transphosphorylation reaction, followed by the hydrolysis of this cyclic intermediate. For this reason, these proteins are known as cyclant RNases.

According to different embodiments, the ribotoxin is a toxin, or an active fragment thereof, selected from the group consisting of
sarcin
restrictocin
anisoplin
hirsutellin
clavin,
mitogillin,
ageritin, and
gigantin.

Ribotoxins have been detected in many different fungi, including entomopathogenic and edible species, but the three-dimensional structure has only been resolved for three of them: α-sarcin, restrictocin, and hirsutellin A (HtA). The first two, produced by Aspergillus giganteus and Aspergillus restrictus, respectively, are nearly identical.

In one embodiment, the ribotoxin is α-Sarcin, or an active fragment thereof.

Different variants of α-Sarcin exist, examples of which are published under the Uni Prot identifiers P00655, Q7LVR0, O14446, O13323, O13324, O13322, O13325, A0A0G2DUB2. While some examples in the present application use P00655 other Sarcin variants can likewise be used. The skilled person can find such variants with routine efforts in the respective databases. One exemplary sequence of Sarcin is given in SEQ ID NO 56, which shows a deimmunized variant thereof. SEQ ID NO 55 shows the wildtype.

In one embodiment, the ribotoxin is hirsutellin A (HtA), or an active fragment thereof. HtA, produced by the entomopathogenic fungus Hirsutella thompsonii, is much smaller and has only shows 25% sequence identity with the other larger ribotoxins. Even so, it retains all the functional characteristics of the family. Different variants of hirsutellin A exist, examples of which are published under the UniProt identifiers N4VY63, P78696, A0A0B4HUA1, A0A0B4FSP6, T5AB58, A0A0B4EQU3, E9FCV0, A0A014PJJ6, A0A0B4GG41, L2G0X6, A0A063C0Y4, A0A179FJ94, A0A166WTA3, A1CDH8, I8AC84, A0A364MLV5, A0A4Q7JNA6, Q8NJP2, Q8NJP0, Q8NJP3, Q8NJP1, Q8NJN9, Q8NIC7, E9E2C8. While some examples in the present application use N4VY63, other hirsutellin A variants can likewise be used. The skilled person can find such variants with routine efforts in the respective databases. One exemplary sequence of hirsutellin A is given in SEQ ID NO 47.

In one embodiment, the ribotoxin is restrictocin (sometimes also called mitogellin), or an active fragment thereof (UniProt identifier: P67876). One exemplary sequence of restrictocin is given in SEQ ID NO 27.

Other variants can likewise be used. The skilled person can find such variants with routine efforts in the respective databases.

In one embodiment, the ribotoxin is clavin, or an active fragment thereof. Different variants clavin exist, examples of which are published under the Uni Prot identifiers, P0CL70, P0CL71, E0YUC8, A0A4R8PRX1, A0A4R8T0U3, U4KU86, A0A4R8R208, U4KUQ3. Other variants can likewise be used. The skilled person can find such variants with routine efforts in the respective databases.

In one embodiment, the ribotoxin is gigantin, or an active fragment thereof (UniProt identifier: P87063). Other variants can likewise be used. The skilled person can find such variants with routine efforts in the respective databases.

In one embodiment, the ribotoxin is anisoplin, or an active fragment thereof (see e.g. SEQ ID NO 48, and a modified variant in SEQ ID NO 49). It is produced by the fungus Metarhizium anisopliae, another insect pathogen.

The term “RNase”, as used herein, relates to a group of nucleases that catalyze the degradation of RNA into smaller components (“Ribonucleases”). Ribonucleases can be divided into endoribonucleases and exoribonucleases, and comprise several sub-classes within the EC 2.7 (for the phosphorolytic enzymes) and 3.1 (for the hydrolytic enzymes) classes of enzymes.

Major types of endoribonucleases as disclosed herein are the following: EC 3.1.27.5: RNase A is an RNase that is commonly used in research. RNase A (e.g., bovine pancreatic ribonuclease A: PDB: 2AAS) is one of the hardiest enzymes in common laboratory usage; one method of isolating it is to boil a crude cellular extract until all enzymes other than RNase A are denatured. It is specific for single-stranded RNAs. It cleaves the 3-end of unpaired C and U residues, ultimately forming a 3′-phosphorylated product via a 2′,3′-cyclic monophosphate intermediate. It does not require any cofactors for its activity EC 3.1.26.4: RNase H is a ribonuclease that cleaves the RNA in a DNA/RNA duplex to produce ssDNA. RNase H is a non-specific endonuclease and catalyzes the cleavage of RNA via a hydrolytic mechanism, aided by an enzyme-bound divalent metal ion. RNase H leaves a 5′-phosphorylated product.

EC 3.1.26.3: RNase III is a type of ribonuclease that cleaves rRNA (16s rRNA and 23s rRNA) from transcribed polycistronic RNA operon in prokaryotes. It also digests double strands RNA (dsRNA)-Dicer family of RNase, cutting pre-miRNA (60-70 bp long) at a specific site and transforming it in miRNA (22-30 bp), that is actively involved in the regulation of transcription and mRNA life-time.

EC number 3.1.26: RNase L is an interferon-induced nuclease that, upon activation, destroys all RNA within the cell

EC 3.1.26.5: RNase P is a type of ribonuclease that is unique in that it is a ribozyme—a ribonucleic acid that acts as a catalyst in the same way as an enzyme. One of its functions is to cleave off a leader sequence from the 5′ end of one stranded pre-tRNA. RNase P is one of two known multiple turnover ribozymes in nature (the other being the ribosome). In bacteria RNase P is also responsible for the catalytic activity of holoenzymes, which consist of an apoenzyme that forms an active enzyme system by combination with a coenzyme and determines the specificity of this system for a substrate. A form of RNase P that is a protein and does not contain RNA has recently been discovered.

EC number 3.1.: RNase PhyM is sequence specific for single-stranded RNAs. It cleaves 3′-end of unpaired A and U residues.

EC 3.1.27.3: RNase T1 is sequence specific for single-stranded RNAs. It cleaves 3′-end of unpaired G residues.

EC 3.1.27.1: RNase T2 is sequence specific for single-stranded RNAs. It cleaves 3′-end of all 4 residues, but preferentially 3′-end of As.

EC 3.1.27.4: RNase U2 is sequence specific for single-stranded RNAs. It cleaves 3′-end of unpaired A residues.

EC 3.1.27.8: RNase V is specific for polyadenine and polyuridine RNA.

EC 3.1.26.12: RNase E is a ribonuclease of plant origin, which modulates SOS responses in bacteria, for a response to the stress of DNA damage by activation of the SOS mechanism by the RecA/LexA dependent signal transduction pathway that transcriptionally depresses a multiplicity of genes leading to transit arrest of cell division as well as initiation of DNA repair.

EC 3.1.26.-: RNase G It is involved in processing the 16′-end of the 5s rRNA. It is related to chromosome separation and cell division. It is considered one of the components of cytoplasmic axial filament bundles. It is also thought that it can regulate the formation of this structure.

Major Types of Exoribonucleases

EC number EC 2.7.7.8: Polynucleotide Phosphorylase (PNPase) functions as an exonuclease as well as a nucleotidyltransferase.

EC number EC 2.7.7.56: RNase PH functions as an exonuclease as well as a nucleotidyltransferase.

EC number 3.1.??: RNase R is a close homolog of RNase II, but it can, unlike RNase II, degrade RNA with secondary structures without help of accessory factors.

EC number EC 3.1.13.5: RNase D is involved in the 3′-to-5′ processing of pre-tRNAs.

EC number 3.1.??: RNase T is the major contributor for the 3′-to-5′ maturation of many stable RNAs.

EC 3.1.13.3: Oligoribonuclease degrades short oligonucleotides to mononucleotides.

EC 3.1.11.1: Exoribonuclease I degrades single-stranded RNA from 5′-to-3′, exists only in eukaryotes.

EC 3.1.13.1: Exoribonuclease II is a close homolog of Exoribonuclease I.

In some embodiments, the RNase is one selected from the following group:
Onconase: (rampirinase, frog rnase): Different variants of Onconase: exist, examples of which are published under the Uni Prot identifiers Q8UVX5, Q9I8V8, Q6EUW9, Q6EUW8, Q6EUW7 or P22069.
RNase 1: Pancreatic ribonuclease (e.g. RNAse1, e.g. Uniprot identifier P07998; see for example SEQ ID NO 57)
RNase 2: Non-secretory ribonuclease (e.g. RNAse2, e.g. Uniprot identifier P10153)
RNase 3: Eosinophil cationic protein (e.g. RNAse3/Drosha, e.g. Uniprot identifier Q9NRR4 or P12724)
RNase 4: Ribonuclease 4 (e.g. RNAse4, e.g. Uniprot identifier P34096)
RNase 5: Angiogenin (e.g. RNAse 5, e.g. Uniprot identifier P03950), see for example SEQ ID NO 50)
RNase 6: Ribonuclease K6/Ribonuclease T2/Ribonuclease K3 (e.g. RNAse6, e.g. Uniprot identifier Q93091)
RNase 7: Ribonuclease 7/Ribonuclease A E1 (e.g. RNAse7, e.g. Uniprot identifier Q9H1E1)
RNase 8: Ribonuclease 8 (e.g. RNAse8, e.g. Uniprot identifier Q8TDE3)

The above Uniprot identifiers have exemplary purpose only. Other variants can likewise be used. The skilled person can find such variants with routine efforts in the respective databases.

In several embodiments, the peptide linker or the cleavable domain in the protoxin is specifically or non-specifically cleavable by an enzyme expressed by a mammalian cell, or an enzyme that is produced by a mammalian host, or is not cleavable by an enzyme expressed by a plant cell, or an enzyme that is produced by a plant host.

In one embodiment, the binder-toxin fusion protein is produced in a plant host or plant cell. As discussed elsewhere, this provides the option to create constructs that have a linker that is cleavable by mammalian enzymes which are not present in plant hosts. In such, self intoxication of the production system is avoided while the cleavable linker allows quick release of the toxin in vivo.

In one embodiment, the binder-toxin fusion protein is produced in a mammalian cell, like e.g CHO.

In one embodiment, the plant host or plant cell is transiently modified by means of a vector encoding, inter alia, the binder-toxin fusion protein.

In one embodiment, the plant host or plant cell is permanently modified by means of a vector encoding, inter alia, the binder-toxin fusion protein.

Background on methods for expressing binder-toxin fusion proteins in plant hosts or plat cells, transiently or permanently, is provided in WO2020169620, the content of which is incorporated herein for enablement purposes.

In one embodiment, the plant host or plant cell is from the genus Nicotiana. In this context, it is again mentioned that in one embodiment, the peptide linker or the cleavable domain of the protoxin is not cleavable by an enzyme expressed by a plant cell, or an enzyme that is produced by a plant host. In such way, the producing plant cell or plant host is protected from self intoxication due to unwanted cleavage of the binder-toxin fusion protein.

In one embodiment, the plant or plant cell with which the nucleic acid construct is contacted is not a chloroplast, or not a chloroplast of an algae, in particular not the chloroplast of Chlamydomonas reinhardtii. In another embodiment, structure in the plant or plant cell with which the nucleic acid construct is contacted is not a chloroplast, or not a chloroplast of an algae, in particular not the chloroplast of Chlamydomonas reinhardtii.

In another embodiment where the protein binder comprises two or more chains it may be provided that two nucleic acid constructs are provided, the first comprising the three polynucleotides encoding for the first chain of the protein binder, the linker and the toxin, while the second comprises the polynucleotide encoding for the second chain of the protein binder.

Both transient and stable expression could be induced by an “inducible promoter”. These promoters selectively express an operably linked DNA sequence following to the presence of an endogenous or exogenous stimulus or in response to chemical, environmental, hormonal, and/or developmental signals. These regulatory elements are, without limitation, sensitive to ethanol, heat, light, stress, jasmone, salicylic acid, phytohormones, salt, flooding or drought, as reviewed by Abdel-Ghany et al (2015) and discussed in U.S. Ser. No. 10/344,290 B2, both of which are incorporated herein by reference. Inducible promotors including, but not limited to, synthetic components discuss in Ali et al (2019), the content of which is incorporated herein by reference.

The genus Nicotiana encompasses tobacco plants. Tobacco plants or plant cells have already been tested to produce recombinant immunotherapeutic binder-toxin fusion proteins composed of a small sFv fragment linked to a protein toxin with a stable linker (Francisco et al. (1997), and U.S. Pat. No. 6,140,075A.

According to one further embodiment of the invention, the plant cell is at least one selected from the group consisting of:
Nicotiana tabacum cv. BY2,
Nicotiana tabacum NT-1,
Arabidopsis thaliana,
Daucus carota, and/or
Oyrza sativa.

Nicotiana tabacum cv. BY2 aka Tobacco BY-2 cells and cv. Nicotiana tabacum 1 (NT-1, a sibling of BY-2) are nongreen, fast growing plant cells which can multiply their numbers up to 100-fold within one week in adequate culture medium and good culture conditions. This cultivar of tobacco is kept as a cell culture and more specifically as cell suspension culture (a specialized population of cells growing in liquid medium, they are raised by scientists in order to study a specific biological property of a plant cell). In cell suspension cultures, each of the cells is floating independently or at most only in short chains in a culture medium. Each of the cells has similar properties to the others.

The model plant system is comparable to HeLa cells for human research. Because the organism is relatively simple and predictable it makes the study of biological processes easier, and can be an intermediate step towards understanding more complex organisms. They are used by plant physiologists and molecular biologists as a model organism, and also used as model systems for higher plants because of their relatively high homogeneity and high growth rate, featuring still general behaviour of plant cell. The diversity of cell types within any part of a naturally grown plant (in vivo) makes it very difficult to investigate and understand some general biochemical phenomena of living plant cells. The transport of a solute in or out of the cell, for example, is difficult to study because the specialized cells in a multicellular organism behave differently. Cell suspension cultures such as tobacco BY-2 provide good model systems for these studies at the level of a single cell and its compartments because tobacco BY-2 cells behave very similarly to one another. The influence of neighboring cells behavior is in the suspension is not as important as it would be in an intact plant. As a result any changes observed after a stimulus is applied can be statistically correlated and it could be decided if these changes are reactions to the stimulus or just merely coincidental. BY-2 and NT-1 cells are relatively well understood and often used in research, including the expression of heterologous proteins, in particular antibodies (Hellwig et al (2004). Such methods are disclosed in Hakkinen et al. (2018), the content of which is incorporated herein by reference.

Torres (1989) discusses methods to establish Carrot Cell Suspension Cultures (Daucus carota). Shaaltiel et al (2007) discuss the production of enzymes using a carrot cell based expression system. The content of these articles is incorporated herein by reference. Daucus carota and Oryza sativa are also discussed as suitable plant-cell based expressions systems in Santos et al (2016), the content of which is incorporated herein by reference. The Production of recombinant proteins in Nicotiana tabacum, Arabidopsis thaliana, Oryza sativa is disclosed in Plasson et al (2009), the content of which is incorporated herein by reference.

Generally, the present invention can be practiced with any plant variety for which cells of the plant can be transformed with an DNA construct suitable for expression of a foreign polypeptide and cultured under standard plant cell culture conditions. Plant cells suspension or plant tissues culture is preferred, although callus culture or other conventional plant cell culture methods may be used.

According to one other embodiment of the invention, the plant is Nicotiana benthamiana. The production of antibodies in Nicotiana plants is for example disclosed in Daniell et al. (2001), the content of which is incorporated herein by reference.

Other plants or plant cells that can be used in the context of the present invention include, but are not limited to, lettuce (Lactuca spp.), spinach (Spinacia oleracea), and Arabidopsis (Arabidopsis spp).

In several embodiments, the cleavage site is selected from the group consisting of
a) Endosomal and/or Lysosomal proteases cleavage site
b) Cytosolic protease cleavage site, and/or
c) Cell surface proteases cleavage site.

Examples of such enzymes and their cleavage sites are shown in the following table (see also Choi et al (2012), the content of which in incorporated by reference herein. Reference is made, in this table, to the “Merops” database for more enabling information as regards the respective enzymes. https://www.ebi.ac.uk/merops/index.shtml.

| | | | cleavage sequence | |
|---|---|---|---|---|
| | | | (one letter code) | |
| | | | general motif (examples only) | |
| | | | X can be any naturally | |
| Class | Enzyme class | example | proteinogenic amino acid | reference |
| “Linker | Proprotein | Furin | RXR/KR↓S/A/G/Nxxx | merops S08.071 |
| class 1” | convertase | | | |
| Endosomal | subtilisin/kexin | | | |
| and/or | family | | | |
| Lysosomal | Cathepsins | Cathepsin B | xxF/xV/R/G/L/S/A↓ A/F/Lxxx | merops C01.060 |
| Cleavage | | Cathepsin E | xxxL/F↓Vxxx | merops A01.010 |
| site | | Cathepsin D | xxxL/F↓xxxx | merops A01.009 |
| | | Cathepsin L | xxL/V/F/IR/K↓S/A/Gxxx | merops C01.032 |
| | | Cathepsin K | xK/R/GF/L/I/V/Px↓xxxx | merops C01.036 |
| | | Cathepsin C | xSxE/S↓xxxG/R | merops C01.070 |
| “Linker | Caspases | Caspase 3 | DxxD↓A/G/S/Txxx Or | merops C14.003 |
| class 2” | | | xxxx↓GGFV | |
| | | Caspase 8 | D/LxxD↓G/S/Axxx | merops C14.009 |
| Cytosolic | Kallikereins | hK 1 | xxF/IR/Y↓R/SxGx | merops S01.160 |
| cleavage | (hK) | hK 2 | G/K/AxxR↓xxxG/S/T | merops S01.161 |
| site | | hK3 | S/IS/QxY/Q/R↓SSxx | |
| | | hK10 | No determined | |
| “Linker | Matrix metallo | MMP2 | xP/Axx↓L/Ixxx | merops M10.003 |
| class 3” | proteases | MMP1 | xP/Axx↓L/Ixx | merops M10.001 |
| Cell | | MMP3 | xxxR/N/G↓L/Kxx | merops S01.072 |
| surface | | MMP7 | xPA/G/Lx↓Lxxx | merops M10.005 |
| cleavage | | MMP8 | GP/A/Sxx↓Lxxx | merops M10.002 |
| site | | MMP9 | GP/Axx↓Lxxx | merops M10.004 |
| | | | P2 is preferably a L | |
| | | | P1 is preferably a G | |
| | | MMP12 | GP/A/GL/A/Gx↓Lxxx | merops M10.009 |
| | | MMP14 | xPxx↓Lxxx | merops M10.014 |
| | Matriptase | Matriptase 2 | xxxR↓k/G/Rxxx | merops S01.308 |
| | | Matriptase 1 | xxxR↓K/V/A/RVxx | merops S01.302 |
| | tissue-type | Urokinase type | xSG/SR/K↓xR/Vxx | merops S01.231 |
| | plasminogen | plasminogen | | |
| | activator | activator (uPA) | | |

The cleavage site is described from the cleavage site point (represented by j). The letter x refers to all amino acids. Where there are several preferential amino acids, there are separated by a slash (/).

Such enzyme is preferably a protease. In one embodiment, said peptide linker is not cleavable by a plant enzyme.

Furin is an enzyme which belongs to the subtilisin-like proprotein convertase family, and cleaves proteins C-terminally of the canonic basic amino acid sequence motif Arg-X-Arg/Lys-Arg (RX(R/K)R), wherein X can be any naturally proteinogenic amino acid. Said motif is called a furin cleavage site herein.

Preferably, the sequence thereof is HRRRKRSLDTS (SEQ ID NO 46, called also Liop or FCS I (“Furin cleavage site 1”) herein). Further cleavable linkers that can be used in the context of the present invention are TRHRQPRGWEQL (SEQ ID NO 44, called also Fpe or FCS II herein) and AGNRVRRSVG (SEQ ID NO 45, called also Fdt or FCS III herein)

Cathepsins are proteases found in all animals as well as other organisms. Most of the members become activated at the low pH found in lysosomes. Cathepsin B is capable of cleaving a peptide sequence which comprises the dipeptide motif Val-Ala (VA). Said motif is called a Cathepsin B cleavage site herein. The skilled artisan finds sufficient enabling information on cathepsins and their cleavage sites in Turk el al (2012), the content of which is incorporated herein by reference.

Caspases (cysteine-aspartic proteases, cysteine aspartases or cysteine-dependent aspartate-directed proteases) are a family of protease enzymes playing essential roles in programmed cell death. Over 1500 caspase substrates have been discovered in the human proteome. The general cleavage motif is DXXD-A/G/S/T, wherein X can be any naturally proteinogenic amino acid. The skilled artisan finds sufficient enabling information on caspases and their cleavage sites in Kumar el al (2014), the content of which is incorporated herein by reference.

Matrix metalloproteinases (MMPs), also known as matrixins, are calcium-dependent zinc-containing endopeptidases; other family members are adamalysins, serralysins, and astacins. Collectively, these enzymes are capable of degrading all kinds of extracellular matrix proteins, but also can process a number of bioactive molecules. The skilled artisan finds sufficient enabling information on Matrix Metallo Proteases and their cleavage sites in Eckard el al (2016), the content of which is incorporated herein by reference.

Generally, the skilled artisan is capable, by routine considerations and literature referral, to select specific cleavage sites that match with the respective mammalian enzyme, to control target specific release of the protein toxin or protoxin. General guidelines to find these cleavage sites are e.g. disclosed in Rawlings (2016).

According to one embodiment of the invention, the protein toxin or protoxin is a de-immunized variant of a native protein toxin. Recombinant methods to de-immunize protein toxins by sequence modification are disclosed, e.g., in Schmohl et al. (2015), or Grinberg and Benhar (2017), the content of which is incorporated by reference herein.

In one embodiment, said protein toxin or protoxin is not toxic to plants or plant cells. The skilled person has a bunch of routine methods at hand to check whether this condition is met.

See e.g., Klaine and Lewis (1995) for an overview, the content of which is incorporated by reference herein.

According to one embodiment of the invention, said protein comprises at least one plant-specific N-glycan. N-glycans are glycans that are linked to the amide group of asparagine (Asn) residues in a protein, mostly in an Asn-X-Thr or Asn-X-Ser (NXT or NXS) motif, where X is any amino acid except proline. Typical plant-specific N-glycans are disclosed in Gomord et al. (2010), and differ significantly from mammalian N-glycan patterns.

It is in this respect important to stress that N-Glycans produced by plants are markedly different from those produced, e.g., in mammals. In particular, N-Glycans produced by tobacco plants have
a Fucose residue conjugated to the proximal N-Acetyl-Glucosamine residue via a α3 glycosidic link (instead of α6 as in mammals)
a Xylose residue conjugated to the proximal Mannose residue via a β2 glycosidic link
two distal N-Acetyl-Glucosamine residues, each of which carry a Fucose residue via a α3 glycosidic link, and a Galactose residue via a β3 glycosidic link (instead of a neuraminic acid in mammals).

On the other hand, proteins recombinantly expressed in e.g. algae often lack any kind of glycosylation. Algae are however capable of expressing IgG shaped antibodies, or antibody fragments having a one or more disulfide bridges.

The major plant-based glycoforms identified are complex type glycans (GnGn/GnGnXF). Other glycoforms (Man5-Man9, GnGnF, GnGnX, MMXF, Man5Gn and GnM(X)(F)) can be detected as well.

According to this nomenclature, MGnX means for example

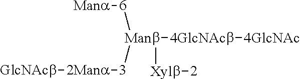

Background on methods for analyzing peptide glycoforms is provided in WO2020169620, the content of which is incorporated herein for enablement purposes.

According to another aspect of the invention, a pharmaceutical composition comprising at least the binder-toxin fusion protein according to the above description is provided, which optionally comprises one or more pharmaceutically acceptable excipients.

According to another aspect of the invention, a combination comprising (i) the binder-toxin fusion protein or the pharmaceutical composition according to the above description, and (ii) one or more further therapeutically active compounds, is provided.

According to another aspect of the invention, the binder-toxin fusion protein, the composition or the combination according to the above description is provided for (the manufacture of a medicament for) use in the treatment of a human or animal subject
suffering from,
being at risk of developing, and/or
being diagnosed for,

developing a neoplastic disease, or for the prevention of such condition.

According to another aspect of the invention, a method for treating a human or animal subject
suffering from,
being at risk of developing, and/or
being diagnosed for

developing a neoplastic disease, or for the prevention of such condition is provided, said method comprising the administration of a therapeutically effective amount of the binder-toxin fusion protein, the composition or the combination according to the above description

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

The examples are based on experiments made with both HPRNAse and Anisoplin, as well as for some other toxins. However, the experimental protocols apply for other toxins belong to or close to those families as well.

The examples are based on experiments made with both furin cleavable linker However, the experimental protocols apply for other sequences sensitive to mammalian enzymes.

Materials and Methods

Genetic Construct Binder-Toxin Fusion Full length Rituximab HC and LC sequences have been used to develop mAb based binder-toxin fusion proteins. Variable parts sequences of the heavy and light chains of rituximab sequences have been assembled in a single chain scFv and fused to a human IgG1 Fc part sequence. A human furin cleavage sequence was then used to fuse the alpha Sarcin sequence at the C-terminal part of the LC or the HC of the full-length rituximab or to the C-terminal part of the scFv-Fc to obtain HC+LC-FCS-alpha Sarcin, HC-FCS-alpha sarcin+LC and scFv-c-FCS-alpha sarcin fusion proteins sequences. Another binder-toxin fusion protein was realized with scFv-Fc part linked to Alpha Sarcin without cleavage site to obtain scFv-Fc-alpha Sarcin. These sequences were produced by gene synthesis flanked with XbaI and IsceI.

Genetic Construct Comprising an Antibody

Full length HC and LC antibody sequences have been used to develop antibody-based binder-toxin fusion proteins. Variable parts sequences of the heavy and light chains of the undisclosed sequences have been assembled in a single chain scFv and fused to a human IgG1 Fc part sequence. A human furin cleavage sequence was then respectively used to fuse the human Anisoplin sequence at the C-terminal part of the LC or the HC or both of the full-length undisclosed antibody or to the C-terminal part of the scFv-Fc to obtain HC+LC-FCS-Anisoplin, HC-FCS-Anisoplin+LC, and scFv-Fc-FCS-Anisoplin or scFv-Fc-Anisoplin fusion proteins sequences. Another binder-toxin fusion protein was realized with scFv-Fc, HC and LC part linked to Anisoplin without cleavage site to obtain scFv-Fc-Anisoplin, HC+LC-Anisoplin, HC-Anisoplin+LC, LC-Anisoplin+HC-Anisoplin. These sequences were produced by gene synthesis flanked with XbaI and IsceI.

Transient Expression in Nicotiana benthamiana Plant Leaves

Nicotiana benthaminana grown under 16 h light/8 h darkness photocycle, 22+/−3° C. 7-8 weeks old plants leaves were transiently transformed by syringe infiltration. Agrobacterium tumefaciens GV3101 (pMP90RK) harboring the undisclosed plasmid containing genetic construct reaching an 600 nm optical density (OD₆₀₀) around 0.8-1.0 were collected by centrifugation at 3500 g for 10 min. Eventually, bacteria were adjusted to an OD₆₀₀ of 0.5 in infiltration buffer (10 mM MgCl2, 10 mM MES, 100 μM acetosyringone, pH 5.6) and the mixture was infiltrated using a needless syringe. Infiltrated regions were harvested 4 and 6 days post agroinfiltration. Entire leaves harvested 4 days post agroinfiltration were used for protein A purification.

Expression in N. tabacum Cells

Nicotiana tabacum plant suspension cells were grown 5 days at 130 rpm, 25° C. in plant culture media as described by Nagata et al. (1992), the content of which is incorporated herein. Agrobacterium tumefaciens LBA4404 (pBBR1MCS-5.virGN54D) harboring the pPZP-ATB binary plasmids reaching an 600 nm optical density (OD₆₀₀) around 0.8-1.0 were collected by centrifugation at 2000 g for 5 min. Plant cells and bacterial cells were then cocultivated in cocultivation media for 30 min before a 2000 g 5 min centrifugation. After supernatant removal, cells were plated on solid cocultivation media for two days. In the case of transient transformation, cells were then collected and washed three times and cultivated in plant cultivation media containing Cefotaxim and Carbeniclin before being harvested for further analysis. In the case of stable transformation, after the 2 days of solid cocultivation, cells were washed and plated on plant media containing selective kanamycin and Cefotaxim and Carbeniclin antibiotics. Callus were selected 4 weeks later and subcultured on solid media or in liquid suspension cultures for subsequent analysis.

Protein Analysis: ELISA, SDS-PAGE and Western Blot

Collected leaves tissues (120 mg) were ground in 400 μL extraction buffer (250 mM Sorbitol, 60 mM Tris, Na2EDTA, 0.6% Polyclar AT, pH8.0). Homogenized tissue was centrifugated at 4° C. for 40 min at 18200 g. Supernatant was then recovered, froze in liquid nitrogen and stored at −20° C.

Extracted tissue were analyzed by western blotting. Proteins were boiled for 5 min in reducing or non-reducing SDS loading buffer (80 mM Tris-HCl, pH 6.8, 2% SDS, 10% glycerol, 0.005% bromophenol blue), centrifuged for 5 min at 13 000 rpm and separated by SDS-PAGE (4-20% polyacrylamide). For Western blotting, proteins were electrotransferred onto a PVDF membrane (Biorad) using a semi-dry electrophoretic device (Biorad Trans-Blot Turbo); then, the membrane was blocked for 1 h at room temperature with 3% (w/v) non-fat milk powder in TBST buffer (50 mM Tris-HCl, 150 mM NaCl, 0.5% Tween 20, pH 7.5) and then incubated (TBS-Tween 0.1%+0.5% non-fat dry milk) for 1 h at room temperature with HRP-conjugated antibodies against the anti-human IgG Fc specific region (A0170; Sigma-Aldrich), at a dilution of 1:10.000 or against Alpha Sarcin primary antibody (in house reagent, anti sarcin rabbit serum, rabbit immunized with alpha-sarcin from Santa Cruz CAS 86243-64-3) at a dilution of 1:10.000. The anti-alpha sarcin/HPRnase antibody was followed by HRP-conjugated anti-rabbit antibodies (0545; Sigma), at a dilution of 1:10 000. Proteins were detected by enhanced chemiluminescence (Amersham Imager 600/GE; GE Healthcare).

Anti CD79b ELISA

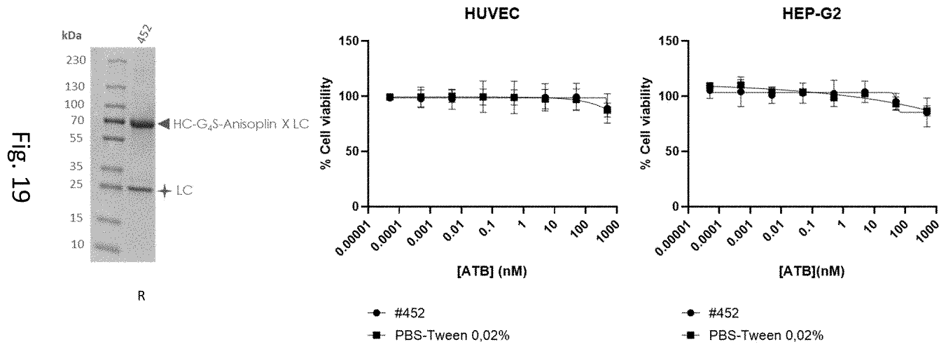

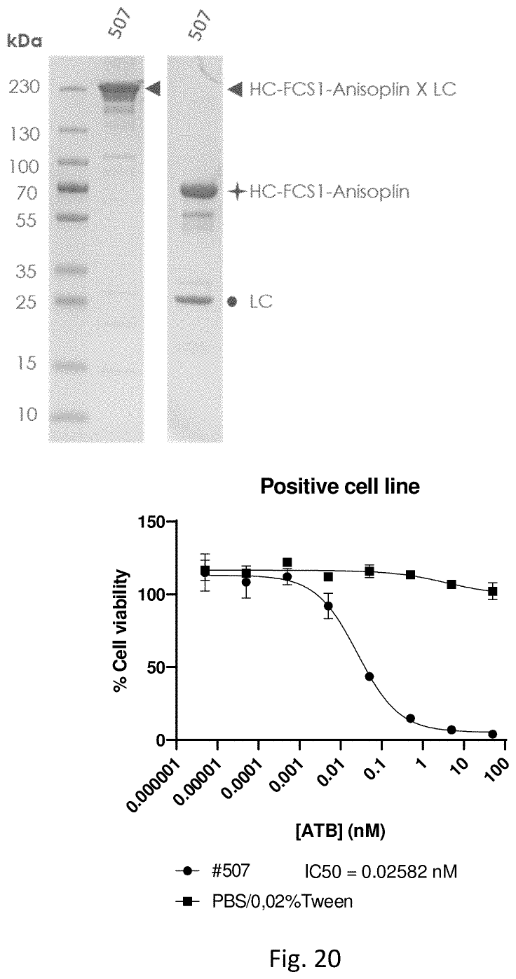

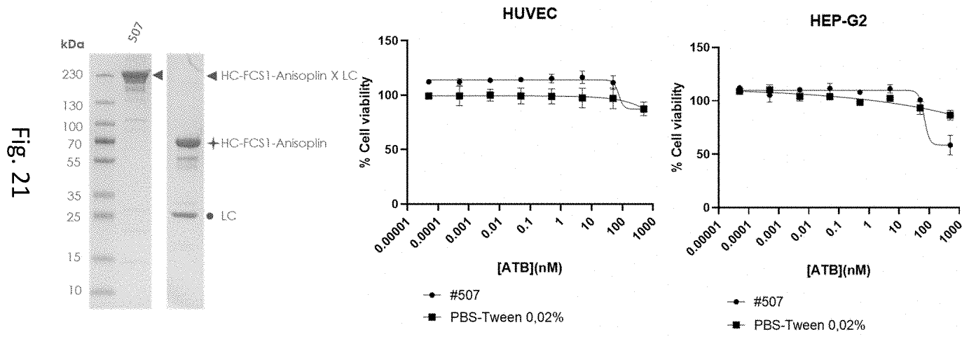

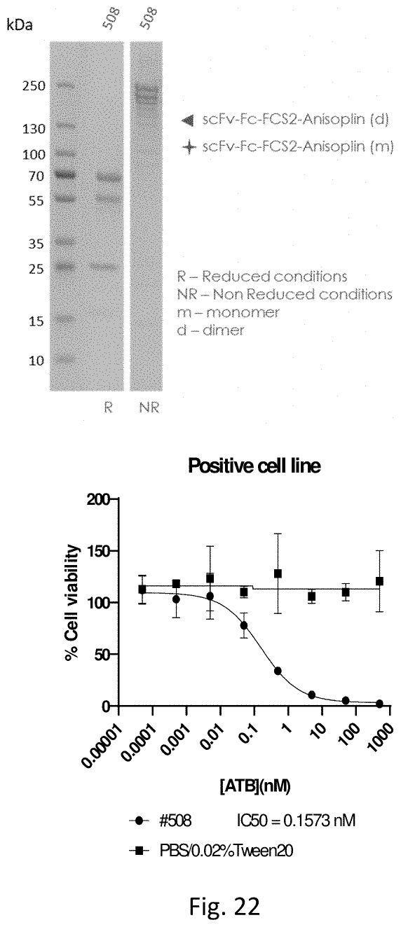

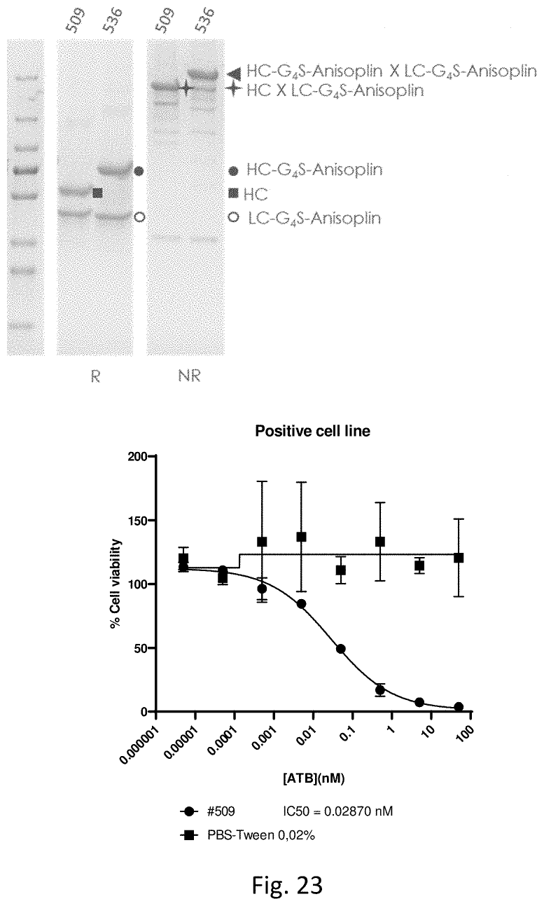

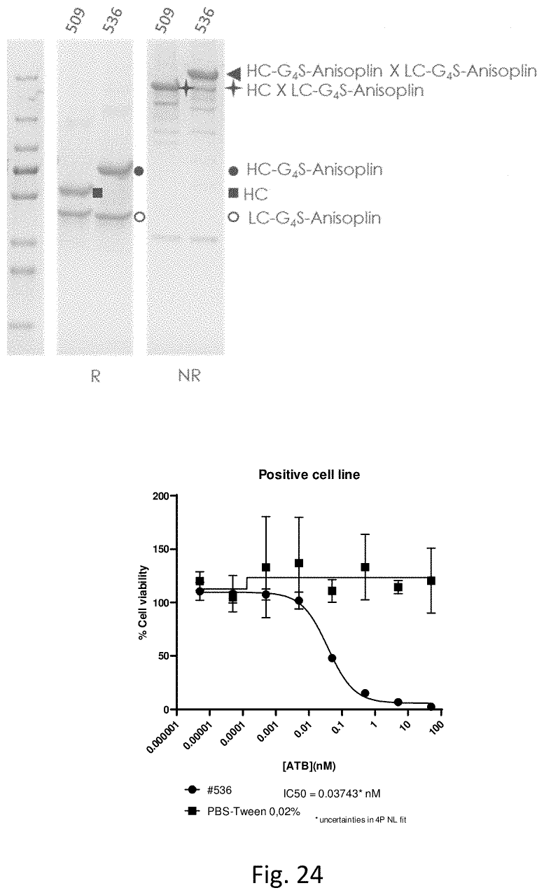

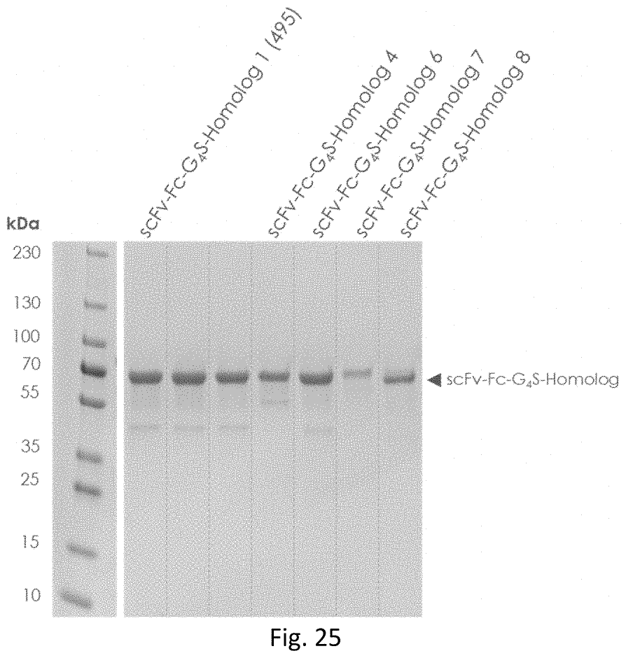

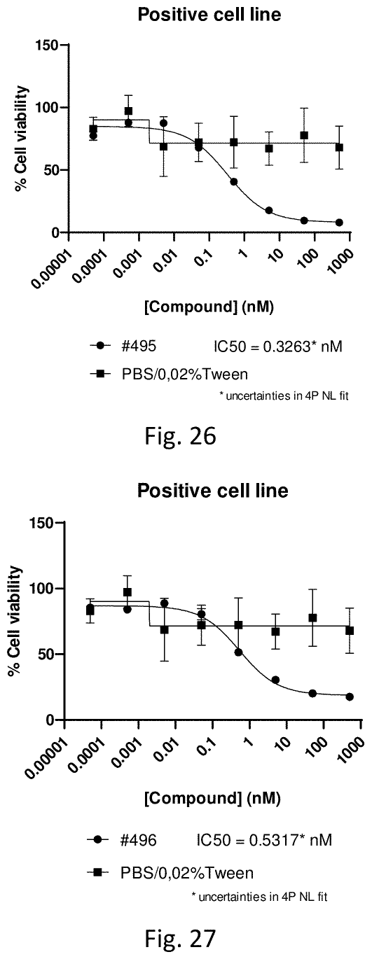

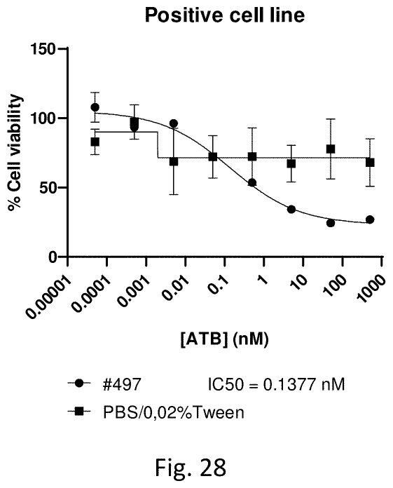

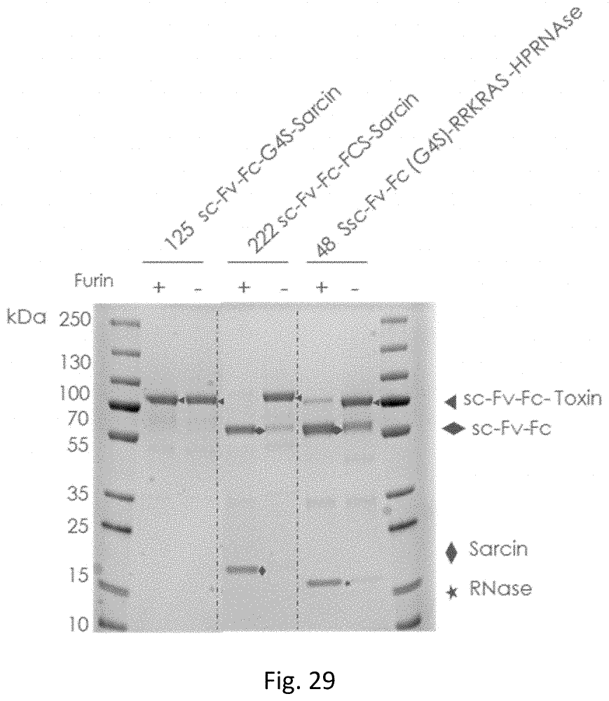

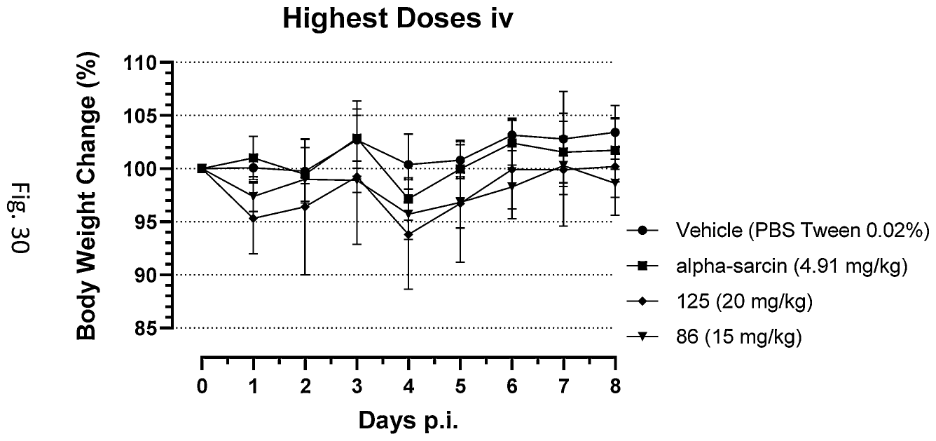

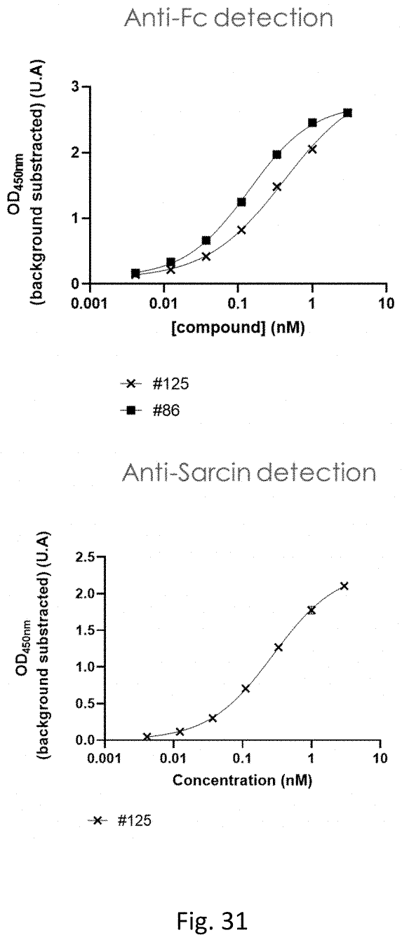

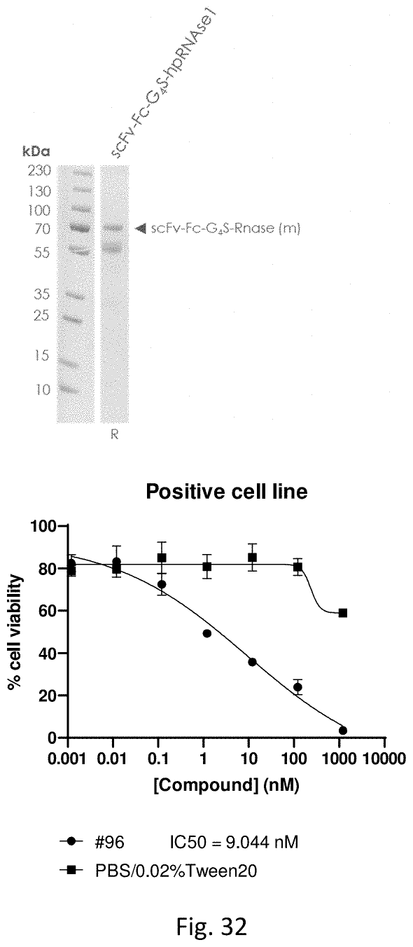

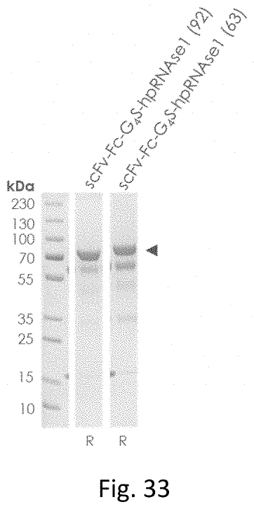

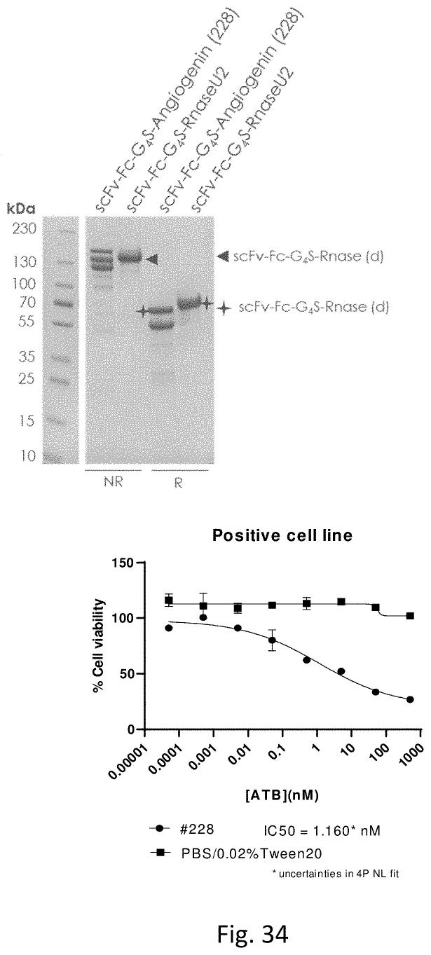

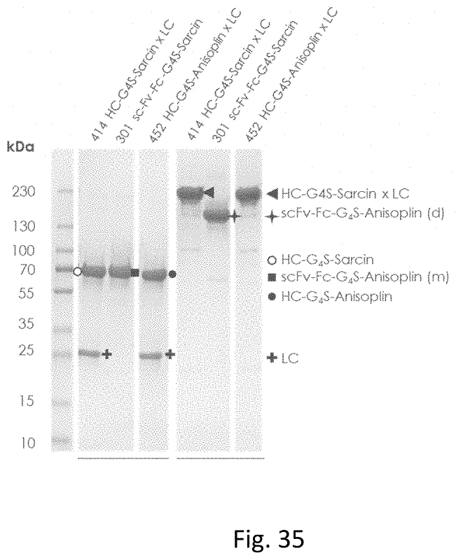

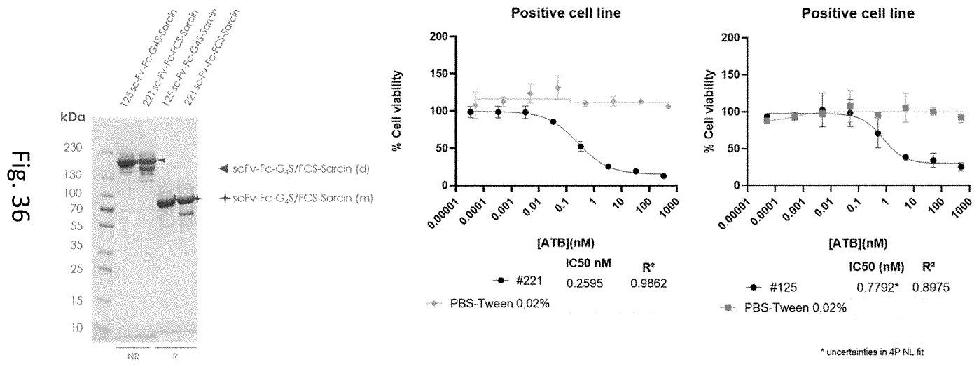

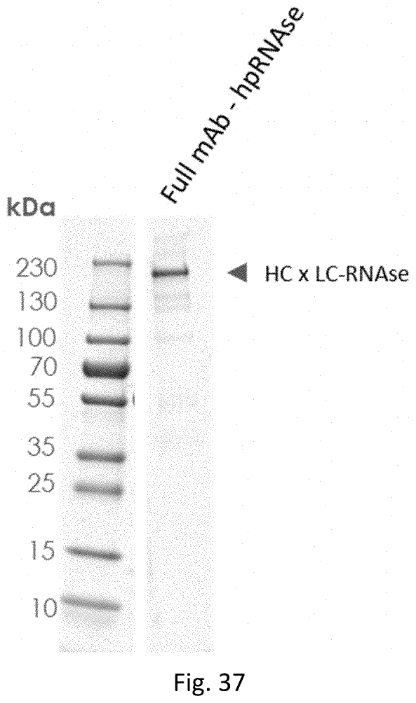

For specificity analysis of a conjugate specific for CD79b, a purified binder-toxin fusion protein comprising a binder against CD79b was analysed by 96 well microplate (Greiner). The wells were coated with 50 μl of antigen CD79b (2.5 μg/mL) for 1 h at 37° C. then washed 5 times with 250 μL washing buffer (PBS Tween 0.1%). Blocking was then performed with 150 μL hydrocasein (3.6%) in PBST for 30 min at RT then washed 5 times. 50 μL anti antigen control antibody was loaded to realize a calibration curve between 5 and 0 μg/mL and 50 μL samples were loaded on the same 96 well plates for comparison for 1 h at RT then washed 5 times. 50 μL of 1/200.000 diluted detection antibody (goat anti-human HRPO, Bethyl) was loaded and incubated 1 h at RT. Revelation was then performed with 50 μL TMB reaction buffer (Zentech) for 15 min and finally stop with H₃PO₄ 1M. Enzymatic activity was then analyzed by spectrometry at 450 nm. Results are shown in FIG. 4B.

Protein a Purification

Four days post agroinfiltration, leaves were collected, weighted and grinded in a blender using 2 mL of extraction buffer (250 mM Sorbitol, 60 mM Tris, Na2EDTA, 0.6% Polyclar AT, pH8.0) per gram of fresh agroinfiltrated leaves. The mixture was then filtered through a double Miracloth (Millipore) layer. The filtrate was then centrifugated at 4° C. for 30 min at 20.000 g. Supernatant was then loaded onto protein A resin preequilibrated with extraction buffer. Resin was then washed with 10 column volume of 60 mM Tris pH8.0 and elution was performed using 100 mM glycine pH3.0 directly buffered with 10% Tris 1M pH8.0. Enriched protein fractions were then collected and freeze in liquid nitrogen.

In Vitro Cytotoxicity Assay

The effect of the binder-toxin fusion proteins on the viability of cell lines expressing CD20 or CD79b was assessed using the Cell Titer Glo Assay (Promega, G9241). In this assay, mono-oxygenation of luciferin is catalyzed by luciferase in presence of Mg 2+ and ATP. This reaction generates a luminescent signal proportional to the number of viable cells.

Depending on the cell line tested, cells were seeded in the cavities of a 96-well plate at a density of 2000 or 5.000 cells/well in 50 μl of growth medium (RPMI1640). Serial dilutions of binder-toxin fusion were prepared by adding 10 μl of binder-toxin fusion or buffer (PBS, Tween 0.02%) to 40 μl of growth medium. The mixture was added to the cells and incubated for 72 hours at 37° C. with 5% CO₂. Binder-toxin fusion were tested in duplicate. Buffer served as a negative control, medium and cells only served as blank and untreated control, respectively.

After 72 hours, plates were equilibrated at room temperature for 30 minutes and 100 μl of CellTiter Glo reagent were added to each well. The plates were subsequently placed on a shaking platform for 2 minutes then signal was allowed to stabilize for 10 minutes at room temperature in the dark. Luminescence was then recorded.

To determine the percentage of viability, the average luminescence signal of the blanks (growth medium only) was subtracted from each well and average luminescence signal of untreated cells was set as 100% viability. The average signal of treated cells was then normalized and plotted as a function of the ATB concentration.

The anti-CD20 based binder-toxin fusion proteins were evaluated on target cells WSU-NHL (CD20+) and non-target cells K562 (CD20−).

The anti-CD79b based binder-toxin fusion proteins were evaluated on target cells JEKO, OCY-LY3, BJAB and WSU-DLCL2 (CD79+) and non-target cells K-562 (CD79−).

In Vivo Assay: Acute Toxicity

To demonstrate the safety of binder-toxin fusion in animals, acute toxicity study was performed on 20 g female NOG mice (Taconic). An undisclosed antibody sc-Fv-Fc-alpha sarcin (125), the alpha sarcin alone and the sc-Fv-Fc (86 as control) have been injected intravenously at respectively at 20 mg/kg, 4.9 mg/kg and 15 mg/kg. Measurement of body weight has been performed each day during 8 consecutive days after injection. The study has been performed by EPO Experimentelle Pharmakologie & Onkologie Berlin-Buch GmbH with material provided by ATB Therapeutics.

Peptide Glycoform Analysis

Background on methods for analyzing peptide glycoforms is provided in WO2020169620, the content of which is incorporated herein for enablement purposes.

Cleavage Assays

Cleavability allowing the release of the toxin have been proved in vitro after addition of recombinant furin on purified scFv-Fc-FCS-alpha sarcin, scFv-Fc-Alpha Sarcin or HPRNAse (binder-toxin fusion protein). The reaction was performed 4 h at 37 degree following addition of 1 μl of 25 units/ml furin (NEB P8077S) to microgram of binder-toxin fusion protein into 15 μl of cleavage buffer (Sodium Acetate 1M pH 5.5+10 mM CaCl₂)). Cleavage have been visualized by SDS Page Coomassie blue gel (4-20% polyacrylamide).

CHO Transient Expression

For control purpose, some constructs 414, 301, 452, 221 and 125 were also expressed in CHO cells. This was done in a vector system developed by Evitria using conventional (non-PCR based) cloning techniques. The evitria vector plasmids were gene synthesized. Plasmid DNA was prepared under low-endotoxin conditions based on anion exchange chromatography. DNA concentration was determined by measuring the absorption at a wavelength of 260 nm. Correctness of the sequences was verified with Sanger sequencing (with up to two sequencing reactions per plasmid depending on the size of the cDNA.)

Suspension-adapted CHO K1 cells were used (originally received from ATCC and adapted to serum-free growth in suspension culture at Evitria) for production. The seed was grown in eviGrow medium, a chemically defined, animal-component free, serum-free medium. Cells were transfected with eviFect, Evitria's custom-made, proprietary transfection reagent, and cells were grown after transfection in eviMake2, an animal-component free, serum-free medium.

Supernatant was harvested by centrifugation and subsequent filtration (0.2 μm filter). The antibody was purified using MabSelect SuRe (Cytivia).

Results

Several recombinant binder-toxin fusion proteins based on the scFv-Fc format have been constructed: scFv-Fc-FCS-Anisoplin, scFv-Fc-Anisopin, scFv-Fc-FCS-HPRNAse, scFv-Fc-HPRNAse, scFv-Fc-FCS-Alpha Sarcin, scFv-Fc-Alpha Sarcin, scFv-Fc-FCS-Alpha sarcin homogs, scFv-Fc-Alpha Sarcin homologues, scFv-Fc-FCS-HPRNAse homologues, and scFv-Fc-HPRNAse homologues. Binder-toxin fusion proteins based on full length mAb have been constructed with Anisoplin, HPRNAse and Alpha sarcin: HC+LC--Anisoplin, HC-Anisoplin or LC, HC-Anisoplin+LC Anisoplin, LC+HC-FCS-Anisoplin or HPRNAse or Alpha Sarcin. Unconjugated mAb alone was also constructed as control.

Cell Viability Assay

Purified binder-toxin fusions have been evaluated on cancer cell line for they cytotoxicity. All binder-toxin fusions have shown to impair positive cell line viability. Moreover, we demonstrated superiority over a marketed ADC (Polivy®, comprising the first humanized anti CD79b antibody (Polatuzumab) kinked to MMAE, as disclosed in U.S. Pat. No. 8,545,850) that target the same antigen (see 425 and 507). In addition, binder-toxin fusion proteins described above have shown very low effect on negative cell line (see 425 and 125).

Binder-toxin fusion proteins are harmless on target negative primary cells HUVEC and HEP2, constating to high efficacy on cancer cells.

Acute Toxicity

Ribotoxin alpha sarcin demonstrated well tolerability in mice when injected at higher dose (4.91 mg/kg). Ribotoxin based binder-toxin fusion, highly active on cancer cells compare to ribotoxin alone, is well tolerated into animal model as IV injection of 20 mg/kg of binder alpha sarcin fusion doesn't trigger any sign of acute toxicity.

CHO Transient Expression

It turned out that constructs 414, 301, 452, 221 and 125 can actually be produced also in CHO cells, albeit at significantly lower yields.

For example, the yield of construct 452 was factor 14-15 lower in the CHO experiment, relative to the Nicotinia experiment. Construct 452 has a G4S linker between the antibody and the toxin, which is not cleavable by mammalian proteases. However, it is still possible, without being bound to theory, that one reason for the lower yield in CHO may be spontaneous cleavage and partial self intoxication occurring in CHO but not in plants like Nicotinia.

Construct 22 has a furin cleavable linker (Fpe), and experiences likewise reduced expression in CHO relative to Nicotinia

| | linker cleavable by | Relative decrease of |
|---|---|---|
| construct | mammalian cell ? | yield in CHO vs Nicotinia |
| 221 (Fpe-SA) | Yes (Fpe) | factor 7-8 |
| 301 (G4S-ANI) | no | factor 8.5-9.5 |
| 452 (Full-G4S-ANI) | no | Factor 14-15 |

Still, it could be shown that construct 221 produced in CHO (which has a furin cleavable linker, Fpe) reduces cell viability in a dose dependent way on positive cell line with an IC₅₀ of 0.2595 nM. Construct 125, which has a non cleavable G4S linker reduces cell viability in a dose dependent way on positive cell line with an IC₅₀ of 0.7792 nM.

Summary of Experimental Results

Experimental results are summarized in the following table.

| | type of | anti- | | | | produc- | | con- | |
|---|---|---|---|---|---|---|---|---|---|
| | exper- | body | antibody | | | tion | | struct | |
| FIG. | iment | target | format | linker | toxin | system | cells | No | Finding |
| 2 | via- | CD20 | sc-Fv-FC | G4S | Sarcin | Nicotiana | WSU-NHL | 206 | ATB 206 reduces cell viability in a |
| | bility | | | | | | | | dose dependent way with an IC50 of |
| | assay | | | | | | | | 0.03669 nM |
| 3 | | CD79b | sc-Fv-FC | G4S | Sarcin | Nicotiana | CD79b + | 86 and | ATB 125 reduces cell viability in a |
| | | | | | | | (JEKO) | 125 | dose dependent way on positive cell |
| | | | | | | | and −(K-562) | | line with an IC50 of 0.3669 nM |
| | | | | | | | | | whereas apha sarcin alone is only |
| | | | | | | | | | active at higher dose with a IC50 of |
| | | | | | | | | | 565 nM. On negative cell line, ATB |
| | | | | | | | | | 125 is not active whereas we |
| | | | | | | | | | observe effect of the alpha sarcin |
| | | | | | | | | | alone at higher dose as for the |
| | | | | | | | | | positive cell line. |
| 4 | | CD79b | sc-Fv-FC | Liop | Sarcin | Nicotiana | CD79b + | 222 | ATB 222 reduces cell viability in a |
| | | | | | | | (BJAB) | | dose dependent way on positive cell |
| | | | | | | | and −(K-562) | | line with an IC50 of 0.000058 nM |
| | | | | | | | | | whereas apha sarcin alone is only |
| | | | | | | | | | active at higher dose with a IC50 of |
| | | | | | | | | | 1.143 nM. On negative cell line, ATB |
| | | | | | | | | | 222 is not active whereas we |
| | | | | | | | | | observe effect of the alpha sarcin |
| | | | | | | | | | alone at higher dose with IC50 of |
| | | | | | | | | | 205.3 nM. |
| 5 | | CD79b | sc-Fv-FC | Fpe | Sarcin | Nicotiana | CD79b + | 221 | ATB 221 reduces cell viability in a |
| | | | | | | | (JEKO) | | dose dependent way on positive cell |
| | | | | | | | | | line with an IC50 of 0.1405 nM. |
| 6 | | CD79b | sc-Fv-FC | Fdt | Sarcin | Nicotiana | CD79b + | 229 | ATB 229 reduces cell viability in a |
| | | | | | | | (JEKO) | | dose dependent way on positive cell |
| | | | | | | | | | line with an IC50 of 0.06368 nM |
| 7 | | CD79b | full IgG | G4S 2x | Sarcin | Nicotiana | CD79b + | 323 | ATB 323 reduces cell viability in a |
| | | | | RRKR | | | (JEKO) | | dose dependent way on positive cell |
| | | | | | | | | | line with an IC50 of 0.1601 nM |
| 8 | | CD79b | sc-Fv-FC | G4S | Ageritin | Nicotiana | CD79b + | 304 | ATB 304 reduces cell viability in a |
| | | | | | | | (JEKO) | | dose dependent way on positive cell |
| | | | | | | | | | line with an IC50 of 0.2807 nM |
| 9 | | CD79b | sc-Fv-FC | G4S | Hirsutelin | Nicotiana | CD79b + | 302 | ATB 304 reduces cell viability in a |
| | | | | | A | | (JEKO) | | dose dependent way on positive cell |
| | | | | | | | | | line with an IC50 of 1.107 nM |
| 10 | | CD79b | sc-Fv-FC | G4S | Anisoplin | Nicotiana | CD79b + | 301 | ATB 301 reduces cell viability in a |
| | | | | | | | (JEKO) | | dose dependent way on positive cell |
| | | | | | | | | | line with an IC50 of 0.1032 nM |
| 11 | | CD79b | sc-Fv-FC | Liop | Anisoplin | Nicotiana | CD79b + | 466 | ATB 466 reduces cell viability in a |
| | | | | | | | (JEKO) | | dose dependent way on positive cell |
| | | | | | | | | | line with an IC50 of 0.0005326 nM |
| 12 | | CD79b | sc-Fv-FC | Fpe | Anisoplin | Nicotiana | CD79b + | 467 | ATB 467 reduces cell viability in a |
| | | | | | | | (JEKO) | | dose dependent way on positive cell |
| | | | | | | | | | line with an IC50 of 0.08602 nM |
| 13 | | CD20 | full IgG | G4S | Anisoplin | Nicotiana | WSU-NHL | 676 | ATB 676 reduces cell viability in a |
| | | | | | | | | | dose dependent way on positive cell |
| | | | | | | | | | line with an IC50 of 0.03760 nM |
| 14 | | CD20 | full IgG | Liop | Anisoplin | Nicotiana | WSU-NHL | 680 | ATB 680 reduces cell viability in a |
| | | | | | | | | | dose dependent way on positive cell |
| | | | | | | | | | line WSU-NHL with an IC50 of |
| | | | | | | | | | 0.01906 nM |
| 15 | | CD79b | full IgG | G4S | Anisoplin | Nicotiana | CD79b + | 452 | ATB 452 reduces cell viability in a |
| | | | | | | | (BJAB) | | dose dependent way on positive cell |
| | | | | | | | and −(K-562) | | line with an IC50 of 0.00003325 nM, |
| | | | | | | | | | a 3000 fold better efficacy |
| | | | | | | | | | compared to a marketed antibody |
| | | | | | | | | | drug conjugated (Polatuzumab) |
| | | | | | | | | | recognizing the same antigen. ATB |
| | | | | | | | | | 452 and benchmark antibody drug |
| | | | | | | | | | conjugate do no reduce viability on |
| | | | | | | | | | negative cell line. |
| 16 | | CD79b | full IgG | G4S | Anisoplin | Nicotiana | CD79b + | 452 | ATB 452 reduces cell viability in a |
| | | | | | | | (JEKO) | | dose dependent way on positive cell |
| | | | | | | | and −(K-562) | | line with an IC50 of 0.01507 nM, a |
| | | | | | | | | | 4.5 fold better efficacy compared to |
| | | | | | | | | | a marketed antibody drug |
| | | | | | | | | | conjugated recognizing the same |
| | | | | | | | | | antigen. The anisoplin alone does |
| | | | | | | | | | not trigger any viability reduction on |
| | | | | | | | | | positive cell line. ATB 452, anisoplin |
| | | | | | | | | | alone and benchmark antibody drug |
| | | | | | | | | | conjugate do no reduce viability on |
| | | | | | | | | | negative cell line. |
| 17 | | CD79b | full IgG | G4S | Anisoplin | Nicotiana | CD79b + | 452 | ATB 452 reduces cell viability in a |
| | | | | | | | (BJAB) | | dose dependent way on positive cell |
| | | | | | | | and −(K-562) | | line with an IC50 of 0.00003325 nM |
| | | | | | | | | | whereas does not trigger |
| | | | | | | | | | cytotoxicity on negative cell line |
| 18 | | CD79b | full IgG | G4S | Anisoplin | Nicotiana | CD79b + | 452 | ATB 452 reduces cell viability in a |
| | | | | | | | (OCY-LY3) | | dose dependent way on positive cell |
| | | | | | | | and −(K-562) | | line with an IC50 of 0.05866 nM |
| | | | | | | | | | whereas does not trigger |
| | | | | | | | | | cytotoxicity on negative cell line |
| 19 | | CD79b | full IgG | G4S | Anisoplin | Nicotiana | Huvec/HepG2 | 452 | ATB 452, Full mab fusion with a non |
| | | | | | | | | | cleavable linker to ribotoxin is well |
| | | | | | | | | | tolerated by human endothelial |
| | | | | | | | | | cells (HUVEC) and human hepatic |
| | | | | | | | | | cells (HEP-G2) |
| 20 | | CD79b | full IgG | Liop | Anisoplin | Nicotiana | CD79b + | 507 | ATB 507 reduces cell viability in a |
| | | | | | | | (JEKO) | | dose dependent way on positive cell |
| | | | | | | | | | line with an IC50 of 0.02582 nM |
| 21 | | CD79b | full IgG | Liop | Anisoplin | Nicotiana | Huvec/HepG2 | 507 | ATB 507, Full mab fusion with a |
| | | | | | | | | | cleavable linker to a ribotoxin is well |
| | | | | | | | | | tolerated by human endothelial |
| | | | | | | | | | cells (HUVEC) and human hepatic |
| | | | | | | | | | cells (HEP-G2) |
| 22 | | CD79b | full IgG | Fpe | Anisoplin | Nicotiana | CD79b + | 508 | ATB 508 reduces cell viability in a |
| | | | | | | | (JEKO) | | dose dependent way on positive cell |
| | | | | | | | | | line with an IC50 of 0.1573 nM |
| 23 | | CD79b | full IgG | G4S | Anisoplin | Nicotiana | CD79b + | 509 | ATB 509 reduces cell viability in a |
| | | | | | | | (JEKO) | | dose dependent way on positive cell |
| | | | | | | | | | line with an IC50 of 0.02870 nM |
| 24 | | CD79b | full IgG | G4S | Anisoplin | Nicotiana | CD79b + | 536 | ATB 536 reduces cell viability in a |
| | | | | | | | (JEKO) | | dose dependent way on positive cell |
| | | | | | | | | | line with an IC50 of 0.03743 nM |
| 25 | | CD79b | sc-Fv-FC | G4S | Anisoplin | Nicotiana | n/a | | |
| | | | | | homologues | | | | |
| | | | | | ONC2 | | | | |
| 26 | | CD79b | sc-Fv-FC | G4S | Anisoplin | Nicotiana | CD79b + | 495 | ATB 495 reduces cell viability in a |
| | | | | | homologue | | (JEKO) | | dose dependent way on positive cell |
| | | | | | 1 | | | | line with an IC50 of 0.3263 nM |
| 27 | | CD79b | sc-Fv-FC | G4S | Anisoplin | Nicotiana | CD79b + | 496 | ATB 496 reduces cell viability in a |
| | | | | | homologue | | (JEKO) | | dose dependent way on positive cell |
| | | | | | 2 | | | | line with an IC50 of 0.5317 nM |
| 28 | | CD79b | sc-Fv-FC | G4S | Anisoplin | Nicotiana | CD79b + | 497 | ATB 497 reduces cell viability in a |
| | | | | | homologue | | (JEKO) | | dose dependent way on positive cell |
| | | | | | 3 | | | | line with an IC50 of 0.1377 nM |
| 29 | cleav- | CD79b | sc-Fv-FC | G4S | Sarcin or | Nicotiana | n/a | 125, 222 | The Furin cleavage assay shows that |
| | age | (sarcin) | | (option- | hpRNase1 | | | and 48 | the payload, either alpha sarcin or |
| | assay | n/d | | ally | | | | | HPRNAse are released from the |
| | | (hPRNase) | | RRKRAS) | | | | | antibody after addition of furin (+) |
| | | | | | | | | | when the linker is cleavable (222 |
| | | | | | | | | | and 48) whereas the non cleavable |
| | | | | | | | | | linker (125) prevents payload |
| | | | | | | | | | release even when exposed to furin. |
| 30 | tox | CD79b | sc-Fv-FC | G4S | Sarcin | Nicotiana | n/a | 125 (86 | Percentage of mean body weight |
| | study | | | | | | | w/o | changes up to 8 days after |
| | | | | | | | | toxin) | intravenous injection of alpha sarcin |
| | | | | | | | | | alone (green), undisclosed sc-Fv-Fc |
| | | | | | | | | | (blue), undisclosed sc-Fv-Fc-alpha |
| | | | | | | | | | sarcin (red). Mean body weight |
| | | | | | | | | | changes don't excess 6%, illustrating |
| | | | | | | | | | good tolerability of binder-toxin |
| | | | | | | | | | fusion based on ribotoxin |
| 31 | Elisa | CD79b | sc-Fv-FC | G4S | Sarcin | Nicotiana | n/a | 125 (86 | Presence of the payload does not |
| | | | | | | | | w/o | modify the binding of the antibody |
| | | | | | | | | toxin) | towards the same coated antigen. |
| 32 | via- | CD79b | sc-Fv-FC | G4S | hpRNase1 | Nicotiana | CD79b + | 96 | ATB 96 reduces cell viability in a |
| | bility | | | | | | (WSU-DLCL2) | | dose dependent way on positive cell |
| | assay | | | | | | | | line with an IC50 of 9.044 nM |
| 33 | gel | CD79b | sc-Fv-FC | G4S | hpRNase1 | Nicotiana | | 92 and | |
| | | | | | | | | 63 | |
| 34 | via- | CD79b | sc-Fv-FC | G4S | Angiogenin | Nicotiana | CD79b + | 228 | ATB 228 reduces cell viability in a |
| | bility | | | | | | (JEKO) | | dose dependent way on positive cell |
| | assay | | | | | | | | line with an IC50 of .160 nM |
| 35 | gel | CD79b | sc-Fv-FC or | G4S | Sarcin or | CHO | n/a | 414, 301, | |
| | | | full IgG | | Anisoplin | | | 452 | |
| 36 | via- | CD79b | sc-Fv-FC | Fpe | Sarcin | CHO | CD79b + | 221 and | ATB 221 reduces cell viability in a |
| | bility | | | | | | (JEKO) | 125 | dose dependent way on positive cell |
| | assay | | | | | | | | line with an IC50 of 0.2595 nM. ATB |
| | | | | | | | | | 125 reduces cell viability in a dose |
| | | | | | | | | | dependent way on positive cell line |
| | | | | | | | | | with an IC50 of 0.7792 nM |
| | | | | | | | | | Production yield in CHO is very |
| | | | | | | | | | small, probably due to partial |
| | | | | | | | | | cleavage of Fpe linker by host |
| | | | | | | | | | enzymes and resulting self |
| | | | | | | | | | intoxication |
| 37 | gel | n/d | IgG | | hpRNase1 | Nicotiana | n/a | | |

### Definitions

“Percentage of sequence identity” as used herein, is determined by comparing two optimally aligned biosequences (amino acid sequences or polynucleotide sequences) over a comparison window, wherein the portion of the corresponding sequence in the comparison window may comprise additions or deletions (i.e., gaps) as compared to the reference sequence, which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides that are substantially identical to the polypeptides exemplified herein. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

The terms “protein toxin” or “protein protoxin”, refer without limitation to toxins that are, by their chemical nature, proteins (i.e., peptides having a length of ≥50 amino acid residues) or polypeptides (i.e., peptides having a length of ≥10-≤50 amino acid residues). A protoxin, in the meaning of the present invention, is a precursor of a toxin, also called a latent toxin, which needs to be activated, e.g., by cleaving off an inhibitory amino acid sequence, or by undergoing a conformational change. The terms “protoxin” and “protein protoxin” are used interchangeably here and mean the same subject matter.

The term “fusion protein” as used herein refers to a protein that has a peptide component operably linked to at least one additional component and that differs from a natural protein in the composition and/or organization of its domains.

The term “operably linked” as used herein, when referring to two or more polynucleotides, means a situation when the different polynucleotides are placed in a functional relationship with one another. For instance, a promoter is operably linked to a coding sequence if the promoter effects the transcription of the coding sequence. Likewise, the coding sequence of a signal peptide is operably linked to the coding sequence of a polypeptide if the signal peptide effects the extracellular secretion of that polypeptide. According to one embodiment of the present invention, when the respective polynucleotides encode different peptides, “operably linked” means that the respective polynucleotides are contiguous and, where necessary to join two protein coding regions, the open reading frames are aligned.

The term “cleavable peptide linker” as used herein refers to an internal amino acid sequence within the fusion protein which contains residues linking the binder moiety and toxin protein so as to render the toxin protein incapable of exerting its toxic effect outside the target cell or limiting its ability of toxin protein to inhibit cell growth (cytostasis) or to cause cell death (cytotoxicity). In such way, the protein toxin is maintained inactive as long as it is in the plasma, until it reaches the target cell, where the cytotoxic payload will be selectively released and/or activated (Grawunder & Stein, 2017). Inside the target cell, the cleavable linker sequence is cleaved and the toxin protein becomes active or toxic. The fusion protein of the invention is composed of a cell-specific binder moiety and an protein toxin moiety linked by a a specific amino acid residue or amino acid sequence that has cleavage recognition site for specific proteases, particularly but not limited to cancer specific protease, and/or are cleavable under specifics conditions such as, without limitation, acid and/or reducing conditions. Sequences encoding cleavage recognition sites for specific protease may be identified among known ubiquitous human protease and/or by testing the expression of cancer associate protease. Also the linker sequence should not interfere with the role of the binder moiety in cell binding and internalization into lysosomes.

The term “cleavable domain” of a protoxin relates to a sequence that, once cleaved by hydrolysis or enzymatic cleavage, activates the toxin part of the protoxin. Many protoxins have an amino acid domain that is specifically cleaved by an enzyme, or by pH dependent hydrolysis (e.g. after endocytosis in the endosomes), so as to release the active toxin part into the cytosol.

Such cleavable domains double act as “naturally occurring” cleavable peptide linkers (or “intrinsic cleavage sites”), contrary to the cleavable peptide linkers which have to be used in case the toxin does not comprise a cleavable domain for activation, e.g., because it does not come as a protoxin.

Hence, while a cleavable linker provides clear advantages over a stable linker as regards the activity profile, the use thereof complicates the production of respective binding protein-toxin conjugates in mammalian, insect and yeast cells, because cleavage of the linker leads to self-intoxication of the production system. This, however, does not apply to plant-based production systems, because
(i) they don't cleave the linker (due to lack of respective proteases or reducing/hydrolyzing conditions) and/or
(ii) the respective protein toxin which is toxic to mammals or mammalian cells is not toxic to plants or plant cells.

As used herein, the term antibody shall refer to an antibody composition having a homogenous antibody population, i.e., a homogeneous population consisting of a whole immunoglobulin, or a fragment or derivative thereof retaining target binding capacities.

Particularly preferred, such antibody is an IgG antibody, or a fragment or derivative thereof retaining target binding capacities. Immunoglobulin G (IgG) is a type of antibody. Representing approximately 75% of serum antibodies in humans, IgG is the most common type of antibody found in blood circulation. IgG molecules are created and released by plasma B cells. Each IgG has two antigen binding sites.

IgG antibodies are large molecules with a molecular weight of about 150 kDa made of four peptide chains. It contains two identical class y heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. The Fc regions of IgGs bear a highly conserved N-glycosylation site. The N-glycans attached to this site are predominantly core-fucosylated diantennary structures of the complex type. In addition, small amounts of these N-glycans also bear bisecting GlcNAc and α-2,6-linked sialic acid residues.

There are four IgG subclasses (IgG1, 2, 3, and 4) in humans, named in order of their abundance in serum (IgG1 being the most abundant).

As used herein, the term “antibody fragment” shall refer to fragments of such antibody retaining target binding capacities, e.g.
a CDR (complementarity determining region),
a hypervariable region,
a variable domain (Fv),
an IgG heavy chain (consisting of VH, CH1, hinge, CH2 and CH3 regions),
an IgG light chain (consisting of VL and CL regions), and/or
a Fab and/or F(ab)2.

As used herein, the term “derivative” shall refer to protein constructs being structurally different from, but still having some structural relationship to the common antibody concept, e.g., scFv, scFv-FC, Fab and/or F(ab)2, as well as bi-, tri- or higher specific antibody constructs or monovalent antibodies, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g. antibody or fragments or derivatives linked to a toxin, a cytokine, a radioisotope or a label). These types are well described in literature and can be used by the skilled person on the basis of the present disclosure, with adding further inventive activity.

Methods for the production of a hybridoma cell have been previously described (see Köhler and Milstein 1975, incorporated herein by reference). Essentially, e.g., a mouse is immunized with a human soluble Guanylyl Cyclase (sGC) protein, followed by B-cell isolation from said mouse and fusion of the isolated B-cell with a myeloma cell.

Methods for the production and/or selection of chimeric or humanized mAbs are known in the art. Essentially, e.g., the protein sequences from the murine anti sGC antibody which are not involved in target binding are replaced by corresponding human sequences. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies. All of these disclosures are incorporated herein by reference.

Methods for the production and/or selection of fully human mAbs are known in the art. These can involve the use of a transgenic animal which is immunized with human sGC, or the use of a suitable display technique, like yeast display, phage display, B-cell display or ribosome display, where antibodies from a library are screened against human sGC in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis. All of these disclosures are incorporated herein by reference.

IgG, scFv, scFv-FC, Fab and/or F(ab)2 are antibody formats well known to the skilled person. Related enabling techniques are available from the respective textbooks.

As used herein, the term “Fab” relates to an IgG fragment comprising the antigen binding region, said fragment being composed of one constant and one variable domain from each heavy and light chain of the antibody.

As used herein, the term “F(ab)2” relates to an IgG fragment consisting of two Fab fragments connected to one another by one or more disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

As used herein, the term “scFv-FC” relates to a specific antibody format. This format is particularly stable and can be expressed with high yield in plant cells and plants. scFv-FC constructs are for example disclosed in Bujak et al (2014), the content of which is incorporated herein by reference. scFv-Fc constructs are dimeric constructs comprising two chains associated to one another for example by one or more disulfide bonds, wherein each of which consist of a structure as follows (in N->C direction):

VL-linker-VH-Linker-FC, or

VH-linker-VL-Linker-FC

with VL being the variable domain of the light chain of an antibody, VH being the variable domain of the heavy chain of an antibody, and FC being the constant domain of an antibody.

The use of a full-length IgG-shaped antibody or a scFv-Fc binding domain confers a longer half-life to the conjugate. Moreover, the Fc part of the antibody might be of utmost importance when CDC (Complement dependent cytotoxicity) or ADCC (Antibody dependent cellular cytotoxicity) activation is required.

Modified antibody formats are for example bi- or trispecific antibody constructs, antibody-based fusion proteins, immunoconjugates and the like. These types are well described in literature and can be used by the skilled person on the basis of the present disclosure, with adding further inventive activity. Furthermore, also monovalent antibodies have been previously described in US 2004/0033561 A1 (referred to therein as monobodies) or WO2007048037; both of which are incorporated herein by reference.

Antibody mimetics are organic compounds—in most cases recombinant proteins or peptides—that, like antibodies, can specifically bind antigens, but that are not structurally related to antibodies. Common advantages over antibodies are better solubility, tissue penetration, stability towards heat and enzymes, and comparatively low production costs. Antibody mimetics are being developed as therapeutic and diagnostic agents, and encompass, inter alia, Affibody molecules, Affilins, Ubiquitins, Affimers, Affitins, Alphabodies, Anticalins, Avimers, DARPins, Fynomers, Kunitz domain peptides, Monobodies and nanoCLAMPs.

Antibody mimetics are discussed in great detail, inter alia, in Gebauer and Skerra (2009), incorporated herein by reference.

Generally, the protein binder may consist of a single chain. This is the case, e.g., where the protein binder is a scFv antibody, or a scFv-FC. In this case, the entire protein binder may be encoded on a single polynucleotide.

In another embodiment the protein binder may comprise two or more chains, like e.g. in a full size IgG or in a F(ab)2 fragment. In such case it may be provided that the nucleic acid construct may comprise two or more polynucleotides encoding for the different chains or domains for the protein binder.

As used herein, the term “plant” (including the cells derived therefrom) relates to algae (including Chlorophyta and Charophyta/Streptophyta, as well as Mesostigmatophyceae, Chlorokybophyceae and Spirotaenia), and also to land plants (Embryophytes), including Gymnospertms and Angiosperms, including Mono- and Dicotyledonae.

As used herein, the term “transient expression” relates to the temporary expression of genes that are expressed for a short time after a nucleic acid, most frequently plasmid DNA encoding an expression cassette, has been introduced into the host cells or plants.

As used herein, the term “stable expression” relates to expression of genes that are expressed continuously in time after a nucleic acid, most frequently plasmid DNA encoding an expression cassette, has been introduced into the host cells' genome (nuclear or plastid integration). In stably transfected cells, the foreign gene becomes part of the genome and is therefore replicated.

### REFERENCES

Abdel-Ghany S. E. (2015). Engineering of plants for the production of commercially important products: approaches and accomplishments. In Plant biology and biotechnology (pp. 551-577). Springer.
Ali, S., & Kim, W. C. (2019). A fruitful decade using synthetic promoters in the improvement of transgenic plants. Frontiers in plant science, 10.
Bujak E et al., Methods Mol Biol. 2014; 1131:315-34
Choi, K Y, et al., Theranostics 2.2 (2012): 156.
Daniell H et al., Trends Plant Sci. 2001 May; 6(5): 219-226.
Eckard U et al, Matrix Biology, Volume 49, January 2016, Pages 37-60
Francisco J A., et al., Binder-toxin fusion protein chemistry 8.5 (1997): 708-713.
Gebauer and Skerra, Curr Opin Chem Biol. 2009 June; 13(3):245-55.
Gomord V et al., (2010). Plant Biotechnology Journal, 8: 564-587
Grinberg Y, Benhar I. Addressing the Immunogenicity of the Cargo and of the Targeting Antibodies with a Focus on Demmunized Bacterial Toxins and on Antibody-Targeted Human Effector Proteins. Biomedicines. 2017 Jun. 2; 5(2):28.
Hakkinen S et al., Front Plant Sci. 2018; 9: 45.
Hashimoto et al. Immunogenetics. 1994; 40(2):145-149.
Klaine, S. J. and M A. Lewis. Algal And Plant Toxicity Testing. 1995 Chapter 8, in Hoffman et al (eds), Handbook of Ecotoxicology. Lewis Publishers, Boca Raton, FL, 163-184, (1995).
Köhler and Milstein, Nature. Bd. 256, S. 495-497
Kumar S et al, PLoS One. 2014; 9(10): e110539
Nagata, T et al., (1992). International Review of Cytology (Vol. 132, pp. 1-30).
Okazaki et al., Blood, 81:84-94 (1993))
Polson et al., Blood. 2007; 110(2):616-623
Rawlings N D, Biochimie, Volume 122, March 2016, Pages 5-30
Santos R et al., Front. Plant Sci., Front. Plant Sci., 11 Mar. 2016
Schmohl J et al, Toxins (Basel). 2015 October; 7(10): 4067-4082.
Shaaltiel, Y et al., (2007) Plant biotechnology journal, 5(5), 579-590
Torres, K. C. (1989). In Tissue Culture Techniques for Horticultural Crops (pp. 161-163). Springer, Boston, MA.)
Turk V et al, Biochimica et Biophysica Acta (BBA)—Proteins and Proteomics, Volume 1824, Issue 1, January 2012, Pages 68-88
Wilbers, R H et al, Plant biotechnology journal, 14(8), 1695-1704

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

Note also that in some embodiments, the respective amino acid sequence has or has not a signal peptide/lead peptide. All embodiments shall be deemed to be disclosed together with the signal peptide/lead peptide and without the signal peptide/lead peptide.

Note also that in some embodiments, the respective amino acid sequence of the toxin shows a deimmunized version thereof. All embodiments shall be deemed to be disclosed with either the wildtype toxin sequence or the deimmunized variant.

| SEQ | | | |
|---|---|---|---|
| ID | antibody | construct | |
| NO | format | No | Sequence |
| 1 | sc-Fv-FC | 206 | QIVLSQSPAILSASPGEKVTMTCRASSSVSYIHWFQQKPGSSPKPWIYATSNL |
| | | | ASGVPVRFSGSGSGTSYSLTISRVEAEDAATYYCQQWTSNPPTFGGGTKLEIK |
| | | | [symbol] |
| | | | QVQLQQPGAELVKPGASVKMSCKASGYTFTSYNMHWVKQTPGRGLEWIGAIYP |
| | | | GNGDTSYNQKFKGKATLTADKSSSTAYMQLSSLTSEDSAVYYCARSTYYGGDW |
| | | | YFNVWGAGTTVTVSA |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 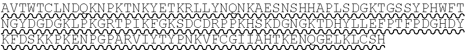 |
| 2 | sc-Fv-FC | 125 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 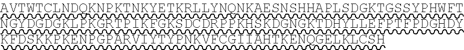 |
| 3 | sc-Fv-FC | 222 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 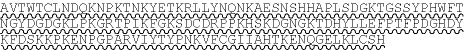 |
| 4 | sc-Fv-FC | 221 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 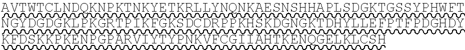 |
| 5 | sc-Fv-FC | 229 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 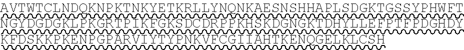 |
| 6 | full IgG | 323, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | [symbol] |
| | | | 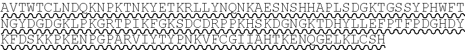 |
| 7 | | 323, LC | QIVLSQSPAILSASPGEKVTMTCRASSSVSYIHWFQQKPGSSPKPWIYATSNL |
| | | | ASGVPVRFSGSGSGTSYSLTISRVEAEDAATYYCQQWTSNPPTFGGGTKLEIK |
| | | | RTVAAPSVFIFPPSDEQLKSGTASVVCLLNNFYPREAKVQWKVDNALQSGNSQ |
| | | | ESVTEQDSKDSTYSLSSTLTLSKADYEKHKVYACEVTHQGLSSPVTKSFNRGE |
| | | | C |
| 8 | sc-Fv-FC | 304 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GGGGS |
| | | | 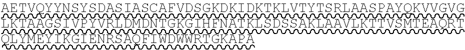 |
| 9 | sc-Fv-FC | 302 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GGGGS |
| | | | 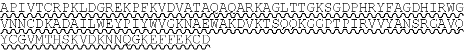 |
| 10 | sc-Fv-FC | 301 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 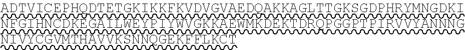 |
| 11 | sc-Fv-FC | 466 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 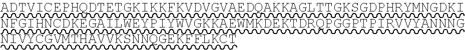 |
| 12 | sc-Fv-FC | 467 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 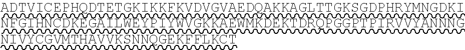 |
| 13 | full IgG | 676, HC | QVQLQQPGAELVKPGASVKMSCKASGYTFTSYNMHWVKQTPGRGLEWIGAIYP |
| | | | GNGDTSYNQKFKGKATLTADKSSSTAYMQLSSLTSEDSAVYYCARSTYYGGDW |
| | | | YFNVWGAGTTVTVSAASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSN |
| | | | TKVDKKAEPKSCDKTHTCPPCPAPELLGGPSVFLFPPKPKDTLMISRTPEVTC |
| | | | VVVDVSHEDPEVKFNWYVDGVEVHNAKTKPREEQYNSTYRVVSVLTVLHQDWL |
| | | | NGKEYKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 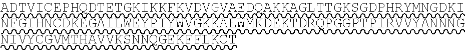 |
| 14 | | 676, LC | QIVLSQSPAILSASPGEKVTMTCRASSSVSYIHWFQQKPGSSPKPWIYATSNL |
| | | | ASGVPVRFSGSGSGTSYSLTISRVEAEDAATYYCQQWTSNPPTFGGGTKLEIK |
| | | | RTVAAPSVFIFPPSDEQLKSGTASVVCLLNNFYPREAKVQWKVDNALQSGNSQ |
| | | | ESVTEQDSKDSTYSLSSTLTLSKADYEKHKVYACEVTHQGLSSPVTKSFNRGE |
| | | | C |
| 15 | full IgG | 680, HC | QVQLQQPGAELVKPGASVKMSCKASGYTFTSYNMHWVKQTPGRGLEWIGAIYP |
| | | | GNGDTSYNQKFKGKATLTADKSSSTAYMQLSSLTSEDSAVYYCARSTYYGGDW |
| | | | YFNVWGAGTTVTVSAASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSN |
| | | | TKVDKKAEPKSCDKTHTCPPCPAPELLGGPSVFLFPPKPKDTLMISRTPEVTC |
| | | | VVVDVSHEDPEVKFNWYVDGVEVHNAKTKPREEQYNSTYRVVSVLTVLHQDWL |
| | | | NGKEYKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 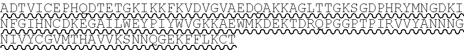 |
| 16 | | 680, LC | QIVLSQSPAILSASPGEKVTMTCRASSSVSYIHWFQQKPGSSPKPWIYATSNL |
| | | | ASGVPVRFSGSGSGTSYSLTISRVEAEDAATYYCQQWTSNPPTFGGGTKLEIK |
| | | | RTVAAPSVFIFPPSDEQLKSGTASVVCLLNNFYPREAKVQWKVDNALQSGNSQ |
| | | | ESVTEQDSKDSTYSLSSTLTLSKADYEKHKVYACEVTHQGLSSPVTKSFNRGE |
| | | | C |
| 17 | full IgG | 452, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVD |
| | | | KKAEPKSCDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKE |
| | | | YKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GGGGS |
| | | | 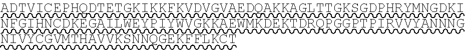 |
| 18 | | 452, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIKRTVAAPSVFIFPPSDEQLKSGTASVVCLLNNFYPREAKVQWKVDNALQ |
| | | | SGNSQESVTEQDSKDSTYSLSSTLTLSKADYEKHKVYACEVTHQGLSSPVTKS |
| | | | FNRGEC |
| 19 | full IgG | 507, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVD |
| | | | KKAEPKSCDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKE |
| | | | YKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 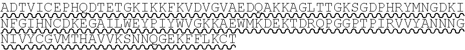 |
| 20 | | 507, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIKRTVAAPSVFIFPPSDEQLKSGTASVVCLLNNFYPREAKVQWKVDNALQ |
| | | | SGNSQESVTEQDSKDSTYSLSSTLTLSKADYEKHKVYACEVTHQGLSSPVTKS |
| | | | FNRGEC |
| 21 | full IgG | 508, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVD |
| | | | KKAEPKSCDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKE |
| | | | YKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 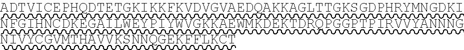 |
| 22 | | 508, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIKRTVAAPSVFIFPPSDEQLKSGTASVVCLLNNFYPREAKVQWKVDNALQ |
| | | | SGNSQESVTEQDSKDSTYSLSSTLTLSKADYEKHKVYACEVTHQGLSSPVTKS |
| | | | FNRGEC |
| 23 | full IgG | 509, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIKRTVAAPSVFIFPPSDEQLKSGTASVVCLLNNFYPREAKVQWKVDNALQ |
| | | | SGNSQESVTEQDSKDSTYSLSSTLTLSKADYEKHKVYACEVTHQGLSSPVTKS |
| | | | FNRGEC |
| | | | [symbol] |
| | | | 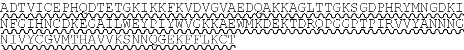 |
| 24 | | 509, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVD |
| | | | KKAEPKSCDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKE |
| | | | YKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| 25 | full IgG | 536, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIKRTVAAPSVFIFPPSDEQLKSGTASVVCLLNNFYPREAKVQWKVDNALQ |
| | | | SGNSQESVTEQDSKDSTYSLSSTLTLSKADYEKHKVYACEVTHQGLSSPVTKS |
| | | | FNRGEC |
| | | | [symbol] |
| | | | 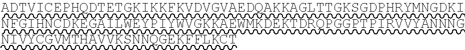 |
| 26 | | 536, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVD |
| | | | KKAEPKSCDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKE |
| | | | YKCKVSNKALPAPIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 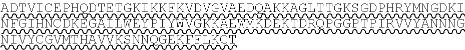 |
| 27 | Mitogillin | | 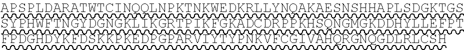 |
| 28 | sc-Fv-FC | 495 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 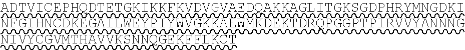 |
| 29 | sc-Fv-FC | 496 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 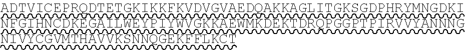 |
| 30 | sc-Fv-FC | 497 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 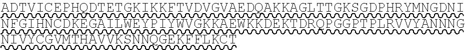 |
| 31 | sc-Fv-FC | 48 | EVQLVESGGGLVKPGGSLKLSCAASGFAFSIYDMSWVRQTPEKRLEWVAYISS |
| | | | GGGTTYYPDTVKGRFTISRDNAKNTLYLQMSSLKSEDTAMYYCARHSGYGTHW |
| | | | GVLFAYWGQGTLVTVSA |
| | | | [symbol] |
| | | | DIQMTQTTSSLSASLGDRVTISCRASQDIHGYLNWYQQKPDGTVKLLIYYTSI |
| | | | LHSGVPSRFSGSGSGTDYSLTISNLEQEDFATYFCQQGNTLPWTFGGGTKLEI |
| | | | K |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | [symbol] |
| | | | 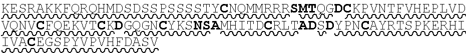 |
| 32 | sc-Fv-FC | 125 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 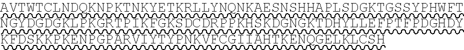 |
| 33 | sc-Fv-FC | 86 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | K |
| 34 | sc-Fv-FC | 96 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | K |
| 35 | sc-Fv-FC | 92 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIKR |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | [symbol] |
| | | | 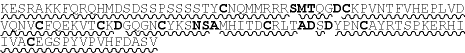 |
| 36 | sc-Fv-FC | 228 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 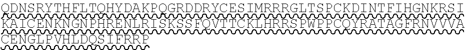 |
| 37 | sc-Fv-FC | 125 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 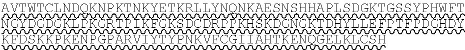 |
| 38 | sc-Fv-FC | 221 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPG |
| | | | [symbol] |
| | | | 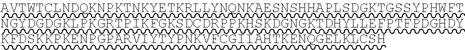 |
| 39 | sc-Fv-FC | 301 | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | [symbol] |
| | | | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | [symbol] |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GGGGS |
| | | | 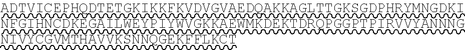 |
| 40 | full IgG | 414, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | ASTKGPSVFPLAPSSKSTSGGTAALGCLVKDYFPEPVTVSWNSGALTSGVHTF |
| | | | PAVLQSSGLYSLSSVVTVPSSSLGTQTYICNVNHKPSNTKVDKKAEPKSCDKT |
| | | | HTCPPCPAPELLGGPSVFLFPPKPKDTLMISRTPEVTCVVVDVSHEDPEVKFN |
| | | | WYVDGVEVHNAKTKPREEQYNSTYRVVSVLTVLHQDWLNGKEYKCKVSNKALP |
| | | | APIEKTISKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 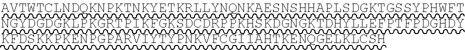 |
| 41 | | 414, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | RTVAAPSVFIFPPSDEQLKSGTASVVCLLNNFYPREAKVQWKVDNALQSGNSQ |
| | | | ESVTEQDSKDSTYSLSSTLTLSKADYEKHKVYACEVTHQGLSSPVTKSFNRGE |
| | | | C |
| 42 | full IgG | 452, HC | EVQLVESGGGLVQPGGSLRLSCAASGYTFSSYWIEWVRQAPGKGLEWIGEILP |
| | | | GGGDTNYNEIFKGRATFSADTSKNTAYLQMNSLRAEDTAVYYCTRRVPIRLDY |
| | | | WGQGTLVTVSS |
| | | | ASTKGPSVFPLAPSSKSTSGGTAALGCLVKDYFPEPVTVSWNSGALTSGVHTF |
| | | | PAVLQSSGLYSLSSVVTVPSSSLGTQTYICNVNHKPSNTKVDKKAEPKSCDKT |
| | | | HTCPPCP |
| | | | APELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTI |
| | | | SKAK |
| | | | GQPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | [symbol] |
| | | | 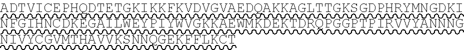 |
| 43 | | 452, LC | DIQLTQSPSSLSASVGDRVTITCKASQSVDYEGDSFLNWYQQKPGKAPKLLIY |
| | | | AASNLESGVPSRFSGSGSGTDFTLTISSLQPEDFATYYCQQSNEDPLTFGQGT |
| | | | KVEIK |
| | | | RTVAAPSVFIFPPSDEQLKSGTASVVCLLNNFYPREAKVQWKVDNALQSGNSQ |
| | | | ESVTEQDSKDSTYSLSSTLTLSKADYEKHKVYACEVTHQGLSSPVTKSFNRGE |
| | | | C |

| | | |
|---|---|---|
| 44 | Fpe liker | TRHRQPRGWEQL |
| 45 | Fdt linker | AGNRVRRSVG |
| 46 | Furin Cleavage site | HRRRKRSLDTS |
| | (FCS)/Liop | |
| 47 | hirsutelin A | 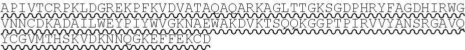 |
| 48 | Anisoplin | 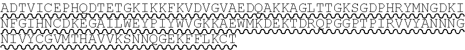 |
| 49 | Anisoplin (version D43Y) | 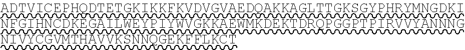 |
| 50 | Angiogenin | 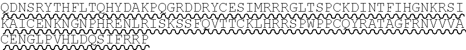 |
| 51 | Anisoplin homologue 1 | 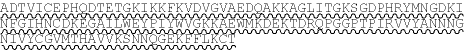 |
| 52 | Anisoplin homologue 2 | 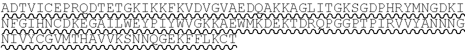 |
| 53 | Anisoplin homologue 3 | 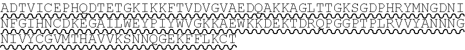 |
| 54 | Ageritin | 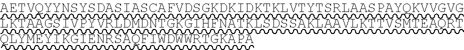 |
| 55 | α-Sarcin | 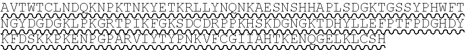 |
| 56 | α-Sarcin (deimmunized version D9T/Q142T) | 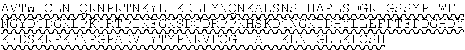 |
| 57 | hpRNase1 | 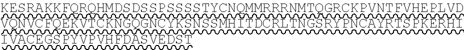 |
| 58 | anti CD79B HCDR1 | GYTFSSYWIE |
| 59 | anti CD79B HCDR2 | GEILPGGGDTNYNEIFKG |
| 60 | anti CD79B HCDR3 | TRRVPIRLDY |
| 61 | anti CD79B LCDR1 | KASQSVDYEGDSFLN |
| 62 | anti CD79B LCDR2 | AASNLES |
| 63 | anti CD79B LCDR3 | CQQSNEDPLT |
| 64 | anti CD20 HCDR1 | YTFTSYNMH |
| 65 | anti CD20 HCDR2 | WIGAIYPGNGDTSY |
| 66 | anti CD20 HCDR3 | RSTYYGGDWYFNV |
| 67 | anti CD20 LCDR1 | SSVSYIH |
| 68 | anti CD20 LCDR2 | PWIYATSNLAS |
| 69 | anti CD20 LCDR3 | QQWTSNPP |

wavy underline = toxin
EVQL . . . = V_{H} domain, underline = HCDR 1-3
DIQL . . . = V_{L} domain, underline = LCDR 1-3
QIVL . . . = V_{L} domain, underline = LCDR 1-3
APELL . . . = CH₂ domain
GQPRE . . . = CH₃ domain
RTVAAP . . . = CL₁ domain
italics = linker or cleavage site

1. A binder-toxin fusion protein comprising anisoplin, or an active fragment thereof, optionally comprising a sequence according to SEQ ID NO 48 or 49, or a homologue thereof having at least 66% sequence identity therewith.

2. The binder-toxin fusion protein according to claim 1, wherein the protein binder is selected from the group consisting of
an antibody
an antibody fragment or derivative retaining target binding capacity, or
an antibody mimetic.

3. The binder-toxin fusion protein according to claim 1, wherein the fusion protein comprises a peptide linker connecting the binder, or a domain thereof, with the toxin, or with a cleavable domain comprised in the toxin.

4. The binder-toxin fusion protein according to claim 1, wherein
the peptide linker or the cleavable domain is specifically or non-specifically cleavable by an enzyme expressed by a mammalian cell, or an enzyme that is produced by a mammalian host, and/or
the peptide linker or the cleavable domain is not cleavable by an enzyme expressed by a plant cell, or an enzyme that is produced by a plant host, and/or
the binder-toxin fusion protein is expressed in a transfected plant cell or transfected whole plant.

5. The binder-toxin fusion protein according to claim 1, wherein the protein binder binds to human CD20 or human CD79B.

6. A binder-toxin fusion protein comprising at least:
a) one protein binder selected from the group consisting of
an antibody
an antibody fragment or derivative retaining target binding capacity, or
an antibody mimetic,
b) a RNAse, a ribotoxin or a respective protoxin, and
c) optionally, a peptide linker the binder, or a domain thereof, with the toxin, or a cleavable domain comprised in the protoxin
wherein the binder-toxin fusion protein is one of the formats selected from the group consisting of
(scFv-FC)-(linker)-toxin (dimer)
tetramer of two HC and two LC-(linker)-toxin
tetramer of two LC and two HC-(linker)-toxin, or
tetramer of two LC-(linker)-toxin and two HC-(linker)-toxin
wherein the linker is optional.

7. A binder-toxin fusion protein comprising at least:
a) one protein binder selected from the group consisting of
an antibody
an antibody fragment or derivative retaining target binding capacity, or
an antibody mimetic,
b) a RNAse, a ribotoxin or a respective protoxin, and
c) optionally, a peptide linker connecting the binder, or a domain thereof, with the toxin, or a cleavable domain comprised in the protoxin
wherein at least one of
the peptide linker or the cleavable domain in the protoxin is specifically or non-specifically cleavable by an enzyme expressed by a mammalian cell, or an enzyme that is produced by a mammalian host, and/or
the peptide linker or the cleavable domain in the protoxin is not cleavable by an enzyme expressed by a plant cell, or an enzyme that is produced by a plant host.

8. A binder-toxin fusion protein comprising at least:
a) one protein binder selected from the group consisting of
an antibody
an antibody fragment or derivative retaining target binding capacity, or
an antibody mimetic,
b) a RNAse, a ribotoxin or a respective protoxin, and
c) optionally, a peptide linker connecting the binder, or a domain thereof, with the toxin, or a cleavable domain comprised in the protoxin
wherein the binder-toxin fusion protein is expressed in a transfected plant cell or transfected whole plant.

9. A binder-toxin fusion protein comprising at least:
a) one protein binder selected from the group consisting of
an antibody
an antibody fragment or derivative retaining target binding capacity, or
an antibody mimetic,
b) a RNAse, a ribotoxin or a respective protoxin, and
c) optionally, a peptide linker connecting the binder, or a domain thereof, with the toxin, or a cleavable domain comprised in the protoxin
wherein the protein binder binds to human CD20 or human CD79b.

10. The binder-toxin fusion protein according to claim 5, wherein the ribotoxin is a toxin, or an active fragment thereof, selected from the group consisting of
sarcin
restrictocin
anisoplin
hirsutellin
clavin,
mitogillin,
ageritin, and
gigantin.

11. The binder-toxin fusion protein according to claim 5, wherein the RNase is a toxin, or an active fragment thereof, selected from the group consisting of
Onconase: rampirinase, frog rnase
RNase 1: Pancreatic ribonuclease (SEQ ID NO 57)
RNase 2: Non-secretory ribonuclease
RNase 3: Eosinophil cationic protein
RNase 4: Ribonuclease 4
RNase 5: Angiogenin (SEQ ID NO 50)
RNase 6: Ribonuclease K6/Ribonuclease T2/Ribonuclease K3
RNase 7: Ribonuclease 7/Ribonuclease A E1, and
RNase 8: Ribonuclease 8.

12. The binder-toxin fusion protein according to claim 1, which binder-toxin fusion protein is produced in a plant host or plant cell.

13. The binder-toxin fusion protein according to claim 1, wherein the plant host or plant cell is from the genus Nicotiana.

14. The binder-toxin fusion protein according to claim 1, wherein the cleavable linker or the cleavable domain in the protoxin comprises at least one cleavage site selected from the group consisting of
a) Endosomal and/or Lysosomal proteases cleavage site
b) Cytosolic protease cleavage site, and/or
c) Cell surface proteases cleavage site.

15. The binder-toxin fusion protein according to claim 1, which protein comprises at least one plant-specific N-glycan.

16. A pharmaceutical composition comprising at least the binder-toxin fusion protein according to claim 1, and optionally one or more pharmaceutically acceptable excipients.

17. A combination comprising (i) the binder-toxin fusion protein according to claim 1 or a pharmaceutical composition, thereof and (ii) one or more further therapeutically active compounds.

18. The binder-toxin fusion protein according to claim 1 a composition or combination thereof for treatment of a human or animal subject
suffering from,
being at risk of developing, and/or
being diagnosed for,
developing a neoplastic disease, or for prevention of such condition.

19. A method for treating a human or animal subject
suffering from,
being at risk of developing, and/or
being diagnosed for
developing a neoplastic disease, or for prevention thereof, said method comprising administration of a therapeutically effective amount of the binder-toxin fusion protein according to claim 1 a composition or combination thereof.
